(19) United States
(12) Patent Application Publication
(10) Pub. No.: US 2016/0081985 A1
(43) Pub. Date: Mar. 24, 2016

(54) CENICRIVIROC COMPOSITIONS AND METHODS OF MAKING AND USING THE SAME

(71) Applicant: Tobira Therapeutics, Inc., South San Francisco, CA (US)

(72) Inventors: Mark Michael Menning, San Francisco, CA (US); Sean Mark Dalziel, Burlingame, CA (US)

(21) Appl. No.: 14/891,019

(22) PCT Filed: May 15, 2014
(86) PCT No.: PCT/US2014/038211

§ 371 Date: Nov. 13, 2015

Related U.S. Application Data
(60) Provisional application No. 61/823,766, filed on May 15, 2013.

(51) Int. Cl. A61K 31/4178; A61K 31/513; A61K 9/24; A61K 9/20; A61K 9/28; A61K 45/06; A61K 31/536

(52) U.S. Cl. CPC A61K 31/4178; A61K 45/06; A61K 31/513; A61K 31/536; A61K 9/2013; A61K 9/28; A61K 9/209

(57) ABSTRACT

The present disclosure relates to pharmaceutical compositions containing cenicriviroc or a salt thereof and optionally one or more additional pharmaceutically active agent, methods for the preparation thereof, and their use in the treatment of diseases or conditions, particularly viruses such as Human Immunodeficiency Virus (HIV).

### CROSS-REFERENCE TO RELATED APPLICATION

This application claims the benefit of priority to U.S. Provisional Application No. 61/823,766, filed May 15, 2013 and entitled “CENICRIVIROC COMPOSITIONS AND METHODS OF MAKING AND USING THE SAME”, the content of which are hereby incorporated by reference in its entirety for all purposes.

### BACKGROUND

1. Field

The present disclosure relates to pharmaceutical compositions containing cenicriviroc or a salt thereof, methods for the preparation thereof, and their use in the treatment of diseases or conditions, particularly viruses such as Human Immunodeficiency Virus (HIV).

2. Background

Cenicriviroc is the common name of (S,E)-8-(4-(2-Butoxyethoxy)phenyl)-1-(2-methylpropyl)-N-(4-(((1-propyl-1H-imidazol-5-yl)methyl)sulfinyl)phenyl)-1,2,3,4-tetrahydrobenzo[b]azocine-5-carboxamide, the chemical structure of which appears in FIG. 1. Cenicriviroc is a weakly basic and poorly water-soluble drug that can be efficacious against viruses, for example retroviruses such as Human Immunodeficiency Virus (HIV). However, clinical use of cenicriviroc can be limited because of bioavailability and stability problems associated with known cenicriviroc compositions. What is more, current cenicriviroc formulations cannot accommodate a daily dose of cenicriviroc in a single tablet, so a subject must take multiple tablets to obtain a sufficient therapeutic effect. Thus, new compositions and formulations comprising cenicriviroc, along with associated methods of making and using such compositions and formulations, are needed. The present invention addresses some of these needs, and provides other related advantages.

### BRIEF SUMMARY

The present disclosure provides, among other things, pharmaceutical compositions containing cenicriviroc as a single active agent or as one of multiple active agents, methods for the preparation thereof, and their use in the treatment of diseases or conditions, particularly viruses such as Human Immunodeficiency Virus (HIV). In certain embodiments, the present compositions are in solid dosage forms. In certain embodiments, the present compositions are oral compositions.

In one embodiment, a composition cenicriviroc or a salt thereof and fumaric acid is provided. In certain embodiments, the cenicriviroc or salt thereof is cenicriviroc mesylate.

In further embodiments, the weight ratio of the cenicriviroc or salt thereof to fumaric acid is from about 7:10 to about 10:7, such as from about 8:10 to about 10:8, from about 9:10 to about 10:9, or from about 95:100 to about 100:95, based on the weight of free cenicriviroc.

In other further embodiments, the fumaric acid is present in an amount of from about 15% to about 40%, such as from about 20% to about 30%, or about 25%, by weight of the composition.

In other further embodiments, the cenicriviroc or salt thereof is present in an amount of from about 15% to about 40%, such as from about 20% to about 30%, or about 25%, by weight of the composition, based on the weight of free cenicriviroc.

In other further embodiments, the composition comprises one or more pharmaceutically inactive ingredients, such as pharmaceutically acceptable excipients, e.g., fillers, disintegrants, lubricants, and etc.

In other further embodiments, the composition comprises one or more fillers. In more specific embodiments, the one or more fillers are selected from microcrystalline cellulose, calcium phosphate dibasic, cellulose, lactose, sucrose, mannitol, sorbitol, starch, and calcium carbonate. For example, in certain embodiments, the one or more fillers is microcrystalline cellulose. In particular embodiments, the weight ratio of the one or more fillers to the cenicriviroc or salt thereof is from about 25:10 to about 10:8, such as from about 20:10 to about 10:10, or about 15:10, based on the weight of free cenicriviroc. In other particular embodiments, the one or more fillers are present in an amount of from about 25% to about 55%, such as from about 30% to about 50% or about 40%, by weight of the composition.

In other further embodiments, the composition further comprises one or more disintegrants. In more specific embodiments, the one or more disintegrants are selected from cross-linked polyvinylpyrrolidone, cross-linked sodium carboxymethyl cellulose, and sodium starch glycolate. For example, in certain embodiments, the one or more disintegrants is cross-linked sodium carboxymethyl cellulose (croscarmellose sodium). In particular embodiments, the weight ratio of the one or more disintegrants to the cenicriviroc or salt thereof is from about 10:10 to about 30:100, such as about 25:100, based on the weight of free cenicriviroc. In other particular embodiments, the one or more disintegrants are present in an amount of from about 2% to about 10%, such as from about 4% to about 8%, or about 6%, by weight of the composition.

In other further embodiments, the composition further comprises one or more lubricants. In more specific embodiments, the one or more lubricants are selected from stearin, magnesium stearate, and stearic acid. For example, in certain embodiments, the one or more lubricants is magnesium stearate. In particular embodiments, the one or more lubricants are present in an amount of from about 0.25% to about 5%, such as from about 0.75% to about 3%, or about 1.25%, by weight of the composition.

In other further embodiments, the composition further comprises one or more anti-tacking agents, such as, e.g., talc. In other further embodiments, the composition further comprises one or more flow aids, such as, e.g., silica.

In other further embodiments, the composition is substantially similar to those described in Table 3a and Table 3b.

In other further embodiments, the composition is substantially similar to that of Example 2b of Table 3a.

In other further embodiments, any of the above-mentioned embodiments is produced by a process involving dry granulation. For example, any of the above-mentioned embodiments may be produced by a process involving dry granulation of an admixture of the cenicriviroc or salt thereof and the fumaric acid.

In other further embodiments, any of the above-mentioned compositions has a water content of no more than about 4% by weight, such as no more than 2% by weight, after six weeks of exposure to about 40° C. at about 75% relative humidity when packaged with desiccant in a container, such as a closed bottle configuration, e.g., an induction sealed bottle.

In other further embodiments, any of the above-mentioned compositions has a total impurity and degradant level of no more than about 2.5%, such as no more than 1.5%, after 12 weeks of exposure to 40° C. at 75% relative humidity when packaged with desiccant in a container, such as a closed bottle configuration, e.g., an induction sealed bottle.

In other further embodiments, the cenicriviroc or salt thereof of any of the above-mentioned compositions has a mean absolute bioavailability after oral administration that is substantially similar to the mean absolute bioavailability of the cenicriviroc or salt thereof in a solution after oral administration. In yet further embodiments, the cenicriviroc or salt thereof has a mean absolute bioavailability of about 10% to about 50%, about 10% to about 30%, about 10% to about 25%, about 15% to about 20%, inclusive of all ranges and subranges therebetween. In a particular embodiment, the cenicriviroc or salt thereof has a mean absolute bioavailability of about 15% to about 20%, inclusive of all ranges and subranges therebetween. In one embodiment, the cenicriviroc or salt thereof has a mean absolute bioavailability of about 13%, 14%, 15%, 16%, 17%, 18%, 19%, 20%, 21%, 22%, 23%, 24%, 25%, 26%, or 27%, inclusive of all ranges and subranges therebetween. In a particular embodiment, the cenicriviroc or salt thereof has a mean absolute bioavailability of about 18%. In a particular embodiment, the aforementioned bioavailability is for the cenicriviroc or salt thereof of any of the above-mentioned compositions in a mammal. In a particular embodiment, the mammal is a dog, such as a beagle dog.

In one embodiment, the present invention provides a pharmaceutical composition comprising about 150 mg of cenicriviroc or a salt thereof, wherein the composition exhibits a steady state AUC₀₋ₗₐₛₜ of about 7,000 h*ng/ml to about 11,000 h*ng/ml, such as from about 7,500 h*ng/ml to about 9,500 h*ng/ml, or from about 8,000 h*ng/ml to about 9,000 h*ng/ml, following administration of the composition to a subject under fed conditions. In one embodiment, the present invention provides a pharmaceutical composition comprising about 150 mg of cenicriviroc or a salt thereof, wherein the composition exhibits a steady state Cₘₐₓ of about 500 ng/ml to about 750 ng/ml, such as from about 550 ng/ml to about 700 ng/ml, following administration of the composition to a subject under fed conditions. In one embodiment, the present invention provides a pharmaceutical composition comprising about 150 mg of cenicriviroc or a salt thereof, wherein the composition exhibits a steady state Cₘᵢₙ of about 100 ng/ml to about 230 ng/ml, such as from about 130 ng/ml to about 200 ng/ml following administration of the composition to a subject under fed conditions.

In another embodiment, the present invention provides a pharmaceutical composition comprising about 200 mg of cenicriviroc or a salt thereof, wherein the composition exhibits an AUC₀₋ₗₐₛₜ of about 13200 h*ng/ml to about 14200 h*ng/ml and a Cₘₐₓ of about 550 ng/ml to about 700 ng/ml following a single dose administration of the composition under fasted conditions.

“Fasted state” or “fasted condition” includes a subject, e.g., a human, who has not consumed any nourishment overnight, such as a subject who has woken up from sleep but not yet eaten or has an empty stomach around bedtime. A subject, particularly a human, in the fasted state can also be a subject who has not consumed any nourishment other than water for at least 6 hours, particularly at least 8 hours, more particularly at least 10 hours, and even more particularly at least 12 hours. “Fed state” or “fed conditions” refers to a subject, e.g., a human, who consumes a one or more of standard meal, a high fat meal, a high-calorie meal, a rice meal, a low-calorie meal, a low-fat meal, a low-carbohydrate meal, and with or without a beverage or drink, such as coffee, tea, water, fruit juice, soda, etc. The meal can be preceded by at least 6, 8, or 10 hours of fasting, for example, 10, 11, or 12 hours of fasting, however, this is not required unless otherwise specified.

In other further embodiments, any of the above-mentioned compositions exhibits an AUC₀₋ₗₐₛₜ of cenicriviroc that is about 175% or more, such as about 200% or more, or about 225% or more, or about 250% or more, of the AUC₀₋ₗₐₛₜ of cenicriviroc exhibited by a reference solid formulation following oral administration. In other further embodiments, any of the above-mentioned compositions exhibits a Cₘₐₓ of cenicriviroc that is at least 40% higher, such as at least 50% higher or at least 55% higher, than the Cₘₐₓ of cenicriviroc exhibited by a reference solid formulation following oral administration. By reference solid formulation, it is meant a solid formulation comprising cenicriviroc or salt thereof and one or more pharmaceutically acceptable excipient but without an acid solubilizer or pH adjusting agent in the formulation.

In other further embodiments, any of the above-mentioned compositions further comprises one or more additional pharmaceutically active agents.

In more specific embodiments, the one or more additional pharmaceutically active agents is one or more antiretroviral drugs selected from CCR5 receptor antagonists, entry inhibitors, nucleoside reverse transcriptase inhibitors, nucleotide reverse transcriptase inhibitors, non-nucleoside reverse transcriptase inhibitors, protease inhibitors, integrase inhibitors, and maturation inhibitors.

In yet further more specific embodiments, the one or more additional pharmaceutically active agents are selected from maraviroc, lamivudine, efavirenz, raltegravir, vivecon, bevirimat, alpha interferon, zidovudine, abacavir, lopinavir, ritonavir, tenofovir, tenofovir disoproxil, tenofovir prodrugs, emtricitabine, elvitegravir, cobicistat darunavir, atazanavir, rilpivirine, and dolutegravir.

In still further more specific embodiments, the one or more additional pharmaceutically active agents include one or more immune system suppressing agents. In yet still further more specific embodiments, the one or more additional pharmaceutically active agents are selected from the group consisting of cyclosporine, tacrolimus, prednisolone, hydrocortisone, sirolimus, everolimus, azathioprine, mycophenolic acid, methotrexate, basiliximab, daclizumab, rituximab, anti-thymocyte globulin, and anti-lymphocite globulin. In other specific embodiments, the one or more additional pharmaceutically active agents are one or more of tacrolimus or methotrexate.

In one embodiment, a composition comprising cenicriviroc or a salt thereof, fumaric acid, and lamivudine (3TC) is provided. In certain embodiments, the cenicriviroc or salt thereof is cenicriviroc mesylate. In further embodiments, the weight ratio of cenicriviroc or salt thereof to lamivudine is from about 1:15 to about 1:1, such as from about 1:12 to about 2:3; about 1:12; about 1:4; or about 1:2 based on the weight of free cenicriviroc. In other further embodiments, lamivudine is present in an amount of from about 25% to about 65%, such as from about 30% to about 60%, about 31.6%; about 33.3%; about 37.5%; about 40.0%; about 46.2%; or about 60% by weight of the composition. In another embodiment, the composition comprises about 15.8% cenicriviroc or salt thereof and about 31.6% lamivudine by weight of the composition and based on the weight of free cenicriviroc. In another embodiment, the composition comprises about 16.7% cenicriviroc or salt thereof and about 33.3% lamivudine by weight of the composition and based on the weight of free cenicriviroc. In another embodiment, the composition comprises about 18.8% cenicriviroc or salt thereof and about 37.5% lamivudine by weight of the composition and based on the weight of free cenicriviroc. In another embodiment, the composition comprises about 20% cenicriviroc or salt thereof and about 40.0% lamivudine by weight of the composition and based on the weight of free cenicriviroc. In another embodiment, the composition comprises about 11.5% cenicriviroc or salt thereof and about 46.2% lamivudine by weight of the composition and based on the weight of free cenicriviroc. In another embodiment, the composition comprises about 5% cenicriviroc or salt thereof and about 60% lamivudine by weight of the composition and based on the weight of free cenicriviroc.

In other further embodiments, the above-described compositions containing cenicriviroc or salt thereof, fumaric acid, and 3TC may further comprise one or more pharmaceutically inactive ingredients, such as pharmaceutically acceptable excipients, e.g., fillers, disintegrants, lubricants, and etc.

In other further embodiments, the above-described compositions containing cenicriviroc or salt thereof, fumaric acid, and 3TC may further comprise one or more fillers. In more specific embodiments, the one or more fillers are selected from microcrystalline cellulose, calcium phosphate dibasic, cellulose, lactose, sucrose, mannitol, sorbitol, starch, and calcium carbonate. For example, in certain embodiments, the one or more fillers is microcrystalline cellulose. In particular embodiments, the weight ratio of the one or more fillers to the cenicriviroc or salt thereof is from about 5:1 to about 1:5, such as from about 1:4 to about 1:5; or from about 2:3 to about 1:2; or from about 2:1 to about 4:3; or from about 5:1 to about 5:2, based on the weight of free cenicriviroc. In other particular embodiments, the one or more fillers are present in an amount of from about 5% to about 30%, such as about 5.8%%; about 6.6%; about 12%; about 20.5%; about 22.2%; about 23.4%; or about 24.8%, by weight of the composition. In another embodiment, the composition comprises about 15.8% cenicriviroc or salt thereof, about 31.6% lamivudine, and 24.8% one or more fillers by weight of the composition and based on the weight of free cenicriviroc. In another embodiment, the composition comprises about 16.7% cenicriviroc or salt thereof, about 33.3% lamivudine, and 23.4% one or more fillers by weight of the composition and based on the weight of free cenicriviroc. In another embodiment, the composition comprises about 18.8% cenicriviroc or salt thereof, about 37.5% lamivudine, and 12.0% one or more fillers by weight of the composition and based on the weight of free cenicriviroc. In another embodiment, the composition comprises about 20% cenicriviroc or salt thereof, about 40.0% lamivudine, and 5.8% one or more fillers by weight of the composition and based on the weight of free cenicriviroc. In another embodiment, the composition comprises about 20% cenicriviroc or salt thereof, about 40.0% lamivudine, and 6.6% one or more fillers by weight of the composition and based on the weight of free cenicriviroc. In another embodiment, the composition comprises about 11.5% cenicriviroc or salt thereof, about 46.2% lamivudine, and 20.5% one or more fillers by weight of the composition and based on the weight of free cenicriviroc. In another embodiment, the composition comprises about 5% cenicriviroc or salt thereof, about 60% lamivudine, and 22.2% one or more fillers by weight of the composition and based on the weight of free cenicriviroc.

In other further embodiments, the above-described compositions containing cenicriviroc or salt thereof, fumaric acid, and 3TC may further comprise one or more disintegrants. In more specific embodiments, the one or more disintegrants are selected from cross-linked polyvinylpyrrolidone, cross-linked sodium carboxymethyl cellulose, and sodium starch glycolate. For example, in certain embodiments, the one or more disintegrants is cross-linked sodium carboxymethyl cellulose. In particular embodiments, the weight ratio of the one or more disintegrants to the cenicriviroc or salt thereof is from about 1:4 to about 3:2, such as about 1:3; about 2:5; about 1:2; or about 1:1, based on the weight of free cenicriviroc. In other particular embodiments, the one or more disintegrants are present in an amount of from about 3% to about 9% by weight of the composition.

In other further embodiments, the above-described compositions containing cenicriviroc or salt thereof, fumaric acid, and 3TC may further comprise one or more lubricants. In more specific embodiments, the one or more lubricants are selected from stearin, magnesium stearate, and stearic acid. For example, in certain embodiments, the one or more lubricants is magnesium stearate. In particular embodiments, the one or more lubricants are present in an amount of from about 0.5% to about 4%, such as from about 0.75% to about 3%, by weight of the composition. In other further embodiments, the composition further comprises one or more anti-tacking agents, such as, e.g., talc. In other further embodiments, the composition further comprises one or more flow aids, such as, e.g., silica.

In other further embodiments, the above-described compositions containing cenicriviroc or salt thereof, fumaric acid, and 3TC is substantially similar to those examples described in Tables 18, 19, 20, 21, 22, 23, and 24.

In other further embodiments, any of the above-described compositions containing cenicriviroc or salt thereof, fumaric acid, and 3TC has a water content of no more than about 4% by weight, such as no more than 2% by weight, after four weeks of exposure to about 40° C. at about 75% relative humidity when packaged with desiccant.

In other further embodiments, any of the above-described compositions containing cenicriviroc or salt thereof, fumaric acid, and 3TC has a total impurity and degradant level of no more than about 4%, such as no more than 2%, after 9 weeks of exposure to 40° C. at 75% relative humidity when packaged with desiccant.

In other further embodiments, any of the above-described compositions containing cenicriviroc or salt thereof, fumaric acid, and 3TC may further comprise efavirenz. In further embodiments, the weight ratio among cenicriviroc or salt thereof, lamivudine, and efavirenz is from about 1:2:4 based on the weight of free cenicriviroc. In other further embodiments, any of the compositions comprises about 10.3% cenicriviroc or salt thereof, about 18.2% lamivudine, and about 36.4% efavirenz by weight of the composition and based on the weight of free cenicriviroc. In other further embodiments, any of the compositions comprises about 9.5% cenicriviroc or salt thereof, about 19.1% lamivudine, and about 38.1% efavirenz by weight of the composition and based on the weight of free cenicriviroc. In other further embodiments, any of the compositions is substantially similar to the examples described in Table 28 or 29. In other further embodiments, any of the compositions has a water content of no more than about 4.0% by weight, such as no more than about 2.0%, after about four weeks of exposure to about 40° C. at about 75% relative humidity when packaged with a desiccant in a container, such as a closed bottle, e.g., an induction sealed bottle. In other further embodiments, any of the compositions has a total impurity and degradant level of no more than about 4.0%, such as no more than about 2.0%, after 9 weeks of exposure to about 40° C. at about 75% when packaged with a desiccant in a container, such as a closed bottle, e.g., an induction sealed bottle.

In one embodiment, the invention provides pharmaceutical formulations comprising any one of the above-mentioned compositions. In one embodiment, the invention provides pharmaceutical formulations comprising cenicriviroc or a salt thereof, lamivudine (3TC), and one or more pharmaceutically acceptable excipients. In another embodiment, the invention provides pharmaceutical formulations comprising cenicriviroc or a salt thereof, efavirenz (EFV), and one or more pharmaceutically acceptable excipients. In yet another embodiment, the invention provides pharmaceutical formulations comprising cenicriviroc or a salt thereof, 3TC, EFV, and one or more pharmaceutically-acceptable excipients. In any of the preceding embodiments, the cenicriviroc or salt thereof is cenicriviroc mesylate.

In one embodiment of the pharmaceutical formulation, the compositions are in form of granulates. In further embodiments, the cenicriviroc or a salt thereof is present in the pharmaceutical composition in the form of a granulate. In some embodiments, the granulate may comprise an acid solubilizer such as fumaric acid. For example, in one embodiment, the cenicriviroc or a salt thereof and fumaric acid are blended with suitable excipients and granulated to obtain granules containing cenicriviroc or salt thereof. The granules containing cenicriviroc or a salt thereof and fumaric acid may be combined with additional excipients to prepare the compositions of the invention. The components present within the granules of cenicriviroc are referred to as “intra-granular” components whereas the components outside of the granules are referred to as “extra-granular” components. In one embodiment, the “intra-granular” components comprise cenicriviroc or salt thereof and fumaric acid; and the “extra-granular” components comprise one or more pharmaceutically active agents, such as 3TC and/or EFV. In other embodiments, the “intra-granular” components comprise cenicriviroc or salt thereof, fumaric acid, and one or more pharmaceutically active agents, such as 3TC and/or EFV; and the “extra-granular” components comprises one or more pharmaceutically active agents other than cenicriviroc or salt thereof, such as 3TC and/or EFV. In other embodiments, the “intra-granular” components comprise cenicriviroc or salt thereof, fumaric acid, and one or more pharmaceutically active agents, such as 3TC and/or EFV; and the “extra-granular” components do not comprise any pharmaceutically active agent.

In another embodiment, a pharmaceutical formulation is provided that comprises a composition of any of the above-mentioned embodiments. In other further embodiments, the composition in the formulation is disposed in a capsule. In other further embodiments, the composition of the formulation is disposed in a sachet. In other further embodiments, the composition of the formulation is a tablet or a component of a tablet.

In still other further embodiments, the composition of the formulation is in one or more layers of a multi-layered tablet. In still other further embodiments, the composition of the formulation is in a single layer tablet.

In one embodiment of a multi-layered tablet, the composition is in a bilayer tablet comprising a single core and a layer outside the single core. In one embodiment of the bilayer tablet, the cenicriviroc or salt thereof and fumaric acid are present in the core; and lamivudine is present in the layer outside the single core. In another embodiment of the bilayer tablet, the cenicriviroc or salt thereof, fumaric acid, and lamivudine are present in the core; and efavirenz is present in the layer outside the single core.

In further embodiments, any of the compositions in the above-mentioned pharmaceutical formulations is substantially similar to the examples described in Table 3a, 36, 18, 19, 20, 21, 22, 23, 24, 28, or 29. In further embodiments, the pharmaceutical formulation is in an oral dosage form, such as a tablet, which contains a composition substantially similar to that of Table 3a, 36, 18, 19, 20, 21, 22, 23, 24, 28, or 29.

In further embodiments, any of the above-mentioned compositions, any of the above-mentioned pharmaceutical formulations, or any of the above-mentioned tablets, is a coated substrate.

In another embodiment, methods for preparing any of the above-mentioned embodiments are provided. In further embodiments, the method comprises admixing cenicriviroc or a salt thereof and fumaric acid to form an admixture, and dry granulating the admixture. In other further embodiments, the method further comprises admixing one or more fillers with the cenicriviroc or salt thereof and fumaric acid to form the admixture. In more specific embodiments, the one or more fillers are selected from microcrystalline cellulose, calcium phosphate dibasic, cellulose, lactose, sucrose, mannitol, sorbitol, starch, and calcium carbonate. For example, in certain embodiments, the one or more fillers is microcrystalline cellulose. In other further embodiments, the method further comprises admixing one or more disintegrants with the cenicriviroc or salt thereof and fumaric acid to form the admixture. In more specific embodiments, the one or more disintegrants are selected from cross-linked polyvinylpyrrolidone, cross-linked sodium carboxymethyl cellulose, and sodium starch glycolate. For example, in certain embodiments, the one or more disintegrants is cross-linked sodium carboxymethyl cellulose. In other further embodiments, the method further comprises admixing one or more lubricants with the cenicriviroc or salt thereof and fumaric acid to form the admixture. In more specific embodiments, the one or more lubricants are selected from stearin, magnesium stearate, and stearic acid. For example, in certain embodiments, the one or more lubricants is magnesium stearate. In other further embodiments, the method further comprises compressing the dry granulated admixture into a tablet. In other further embodiments, the method comprises filling a capsule with the dry granulated admixture.

In other further embodiments, the method further comprises mixing the dry granulated admixture with one or more extragranular materials. In more specific embodiments, the one or more extragranular materials is one or more additional pharmaceutically active agents. In other more specific embodiments, the one or more pharmaceutically active agents is one or more additional antiretroviral drugs. In other more specific embodiments, the one or more additional antiretroviral drugs are selected from CCR5 receptor antagonists, entry inhibitors, nucleoside reverse transcriptase inhibitors, nucleotide reverse transcriptase inhibitors, non-nucleoside reverse transcriptase inhibitors, protease inhibitors, integrase inhibitors, and maturation inhibitors. In other more specific embodiments, the one or more additional antiretroviral drugs are selected from one or more of maraviroc, lamivudine, efavirenz, raltegravir, vivecon, bevirimat, alpha interferon, zidovudine, abacavir, lopinavir, ritonavir, tenofovir, tenofovir disoproxil, tenofovir prodrugs, emtricitabine, elvitegravir, cobicistat darunavir, atazanavir, rilpivirine, and dolutegravir. In still further more specific embodiments, the one or more additional pharmaceutically active agents include one or more immune system suppressing agents. In yet still further more specific embodiments, the one or more additional pharmaceutically active agents are selected from the group consisting of cyclosporine, tacrolimus, prednisolone, hydrocortisone, sirolimus, everolimus, azathioprine, mycophenolic acid, methotrexate, basiliximab, daclizumab, rituximab, anti-thymocyte globulin, and anti-lymphocite globulin. In other specific embodiments, the one or more additional pharmaceutically active agents are one or more of tacrolimus or methotrexate.

In certain embodiments, a portion of the additional pharmaceutically active agent may be added intra-granularly along with cenicriviroc or a salt thereof.

In another embodiment, a method of administering cenicriviroc or a salt thereof is provided comprising administering a composition, formulation, tablet, or composition produced by the method of any of the above-mentioned embodiments. In another embodiment, a method of treating a disease, disorder, or condition is provided comprising administering a therapeutically effective amount of a composition, formulation, tablet, or composition produced by any of the above-mentioned embodiments. In further embodiments, the disease, disorder, or condition is a viral infection. In other further embodiments, the viral infection is a retroviral infection. In other further embodiments, the disease, condition, or disorder is hepatitis, human immunodeficiency virus, or a sarcoma virus. In certain embodiments, the disease, condition, or disorder is human immunodeficiency virus. In additional embodiments, the disease, disorder, or condition is inflammation. In further additional embodiments, the disease, disorder or condition is graft versus host disease, diabetic inflammation, cardiovascular inflammation, or fibrosis.

Further embodiments of the present invention will be apparent to a person of ordinary skill in the art from the following description and examples.

### BRIEF DESCRIPTION OF THE DRAWINGS

FIG. 1 is the chemical formula of cenicriviroc.

FIG. 2 is a graph comparing the absolute bioavailability, in beagle dogs, of cenicriviroc mesylate compounded as an oral solution with that of cenicriviroc mesylate prepared by wet granulation and mixed with various acid solubilizer excipients.

FIG. 3 is a graph of the total impurity and degradant content of different cenicriviroc formulations subjected to accelerated stability testing at 40° C. and 75% relative humidity when packaged with a desiccant in an induction sealed bottle.

FIG. 4 shows the dissolution profile of cenicriviroc from tablets after storage at 40° C. and 75% relative humidity.

FIG. 5 is a dynamic vapor sorption isotherm for different cenicriviroc formulations.

FIG. 6 shows the absorption of cenicriviroc from different formulations at three pre-treatment states in beagle dogs.

FIGS. 7 and 8 show the dissolution profile and disintegration profile, respectively, of tablets of Examples 2a-2e.

FIG. 9 shows the beagle dog absolute bioavailabilities of tablets of Examples 2a-2e.

FIG. 10 shows the compressibility profile of milled granules of Examples 14 and 15.

FIG. 11 shows the compressibility profile of milled granules of Example 14 when compressed using different roller compactors.

FIG. 12 shows the compressibility profile of powder blends of Examples 17, 19, and 20.

FIG. 13 shows the dissolution characteristics of tablets of Example 28 after 4 weeks of storage at 40° C./75% RH. Panel A shows the dissolution profile for 3TC, panel B shows the dissolution profile for CVC, and panel C shows the dissolution profile for EFV.

FIG. 14 shows the dissolution characteristics of tablets of Example 29 after 4 weeks of storage at 40° C./75% RH. Panel A shows the dissolution profile for 3TC, panel B shows the dissolution profile for CVC, and panel C shows the dissolution profile for EFV.

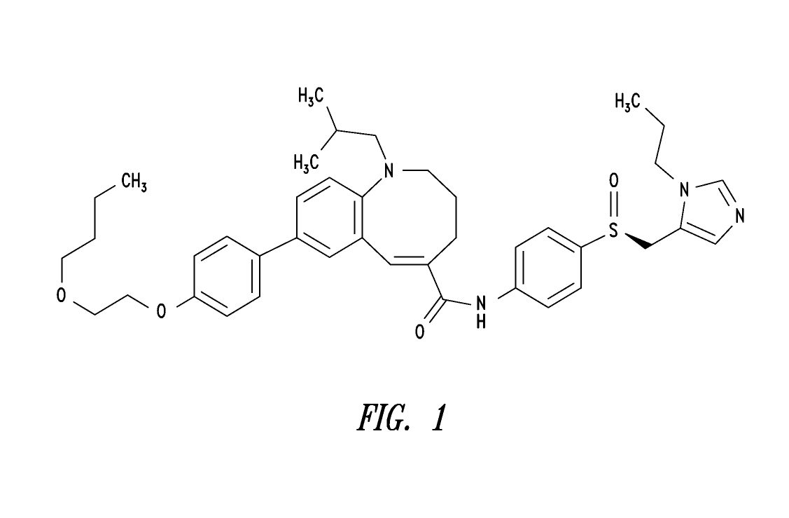

### DETAILED DESCRIPTION

Except where noted, all terms are intended to have their normal meaning in the art, and are used as they would have been used by a person of ordinary skill at the time of the disclosure. It should be understood that throughout this application the singular forms, such as “a,” “an,” and “the,” are often used for convenience, however, these singular forms are intended to encompass the plural unless otherwise specified, or unless the context clearly calls for the singular alone. It should also be understood that all publication, patents, books, journal articles, and the like, which are referred to in this application, are incorporated by reference in their entirety and for all purposes to the extent not inconsistent with the present disclosure.

### DEFINITIONS

“Cenicriviroc” (also known as CVC) refers to the chemical compound (S,E)-8-(4-(2-Butoxyethoxy)phenyl)-1-(2-methylpropyl)-N-(4-(((1-propyl-1H-imidazol-5-yl)methyl)sulfinyl)phenyl)-1,2,3,4-tetrahydrobenzo[1)]azocine-5-carboxamide, which also has the chemical name of 8-[4-(2-butoxyethoxy)phenyl]-1,2,3,4-tetrahydro-1-(2-methylpropyl)-N-[4-[(5)-[(1-propyl-1H-imidazol-5-yl)methyl]sulfinyl]phenyl]-1-Benzazocine-5-carboxamide. Cenicriviroc also has a CAS registry number of 497223-25-3. In certain embodiments, CVC forms acid addition salts, such as a salt of methanesulfonic acid. In one embodiment, the present compositions contain cenicriviroc mesylate.

“Substantially similar” means a composition or formulation that resembles the reference composition or formulation to a great degree in both the identities and amounts of the composition or formulation.

“About” means having a value that is sufficiently close to the reference value so as to have identical or substantially identical properties as the reference value. Thus, depending on context, “about” can mean, for example, ±5%, ±4%, ±3%, ±2%, ±1%, or ± less than 1%.

“Pharmaceutically acceptable” refers to a material or method that can be used in medicine or pharmacy, including for veterinary purposes, for example, in administration to a subject.

“Salt” and “pharmaceutically acceptable salt” includes both acid and base addition salts. “Acid addition salt” refers to those salts that retain the biological effectiveness and properties of the free bases, which are not biologically or otherwise undesirable, and which are formed with inorganic acids and organic acids. “Base addition salt” refers to those salts that retain the biological effectiveness and properties of the free acids, which are not biologically or otherwise undesirable, and which are prepared from addition of an inorganic base or an organic base to the free acid.

“Pharmaceutical formulation” refers to a formulation of a compound of the disclosure and a medium generally accepted in the art for the delivery of the biologically active compound to mammals, e.g., humans. Such a medium includes all pharmaceutically acceptable carriers, diluents or excipients therefor. The pharmaceutical formulations as described herein may be in various dosage forms, such as oral or solid or both dosage forms. In some embodiments, the present pharmaceutical formulations are in tablet or capsule dosage forms.

“Treating” includes ameliorating, mitigating, and reducing the instances of a disease or condition, or the symptoms of a disease or condition. Because the instances of many diseases or conditions can be reduced before the disease or condition manifests, treating can also include prophylaxis.

“Administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, and transdermal. “Administering” can also include prescribing or filling a prescription for a dosage form comprising a particular compound. “Administering” can also include providing directions to carry out a method involving a particular compound or a dosage form comprising the compound.

“Therapeutically effective amount” means the amount of an active substance that, when administered to a subject for treating a disease, disorder, or other undesirable medical condition, is sufficient to have a beneficial effect with respect to that disease, disorder, or condition. The therapeutically effective amount will vary depending on the chemical identity and formulation form of the active substance, the disease or condition and its severity, and the age, weight, and other relevant characteristics of the patient to be treated. Determining the therapeutically effective amount of a given active substance is within the ordinary skill of the art and typically requires no more than routine experimentation.

As noted above, the present disclosure provides a composition, such as a solid composition, containing cenicriviroc or a salt thereof and fumaric acid. The cenicriviroc or salt thereof can be cenicriviroc mesylate. The weight ratio between the cenicriviroc or a salt thereof and fumaric acid, based on the weight of free cenicriviroc, can be from about 7:10 to about 10:7, such as from about 8:10 to about 10:8, from about 9:10 to about 10:9, or from about 95:100 to about 100:95. The fumaric acid can be present in an amount of from about 15% to about 40%, such as from about 20% to about 30%, or about 25%, by weight of the composition. The cenicriviroc or salt thereof can be present, based on the weight of free cenicriviroc, from about 15% to about 40%, such as from about 20% to about 30%, or about 25%, by weight of the composition.

The fumaric acid in the composition can both act as a solubilizer and impart beneficial properties to the composition. For example, fumaric acid can increase the bioavailability of the composition when compared with compositions using other solubilizers, particularly citric acid, maleic acid, and sodium bisulfate.

In some cases, the bioavailability of compositions comprising cenicriviroc mesylate with fumaric acid can approach that of an oral solution. Absorption of an oral solution is not impaired by the rate or extent of drug dissolution. Thus, absorption of drug from a solution is limited only by interactions between the dissolved drug, the body, and ingested materials such as food, beverages, and other drugs. Thus, compositions that approach or equal the bioavailability of an oral solution can be particularly desirable.

This result is surprising and unexpected. As shown in Table 1, fumaric acid has a much lower dissolution time that other acids. Rapidly dissolving acidic excipients were previously believed to have higher solubilizing power on the theory that the excipient should dissolve as fast or faster than the active pharmaceutical ingredient. Several journal articles argue that fumaric acid specifically should not be used in oral dosage forms because of its low solubility and long dissolution time. Thus, it is surprising that the long dissolution time of fumaric acid is associated with higher cenicriviroc bioavailability.

The results described in Table 1 were performed by adding 200 mg of the acid to 90 mL purified water using a Mettler Toledo mixing chamber held at the specified temperature with an upward pumping four blade impeller at 250 rpm. The disappearance of particles undergoing dissolution was monitored by focused-beam reflectance measurement (FBRM). Data was analyzed by reviewing individual 2 second measurement trends as well as trends averaged over 10 and 30 seconds.

TABLE 1

Dissolution time (seconds)

| Acid | 25° C. | 37° C. |
|---|---|---|
| Adipic | 68 | 32 |
| Citric | 6 | <2 |
| Fumaric | 312 | 152 |
| Maleic | 4 | <2 |
| Sodium Bisulfate | 26 | <2 |
| Succinic | 46 | 8 |
| Tartaric | 6 | <2 |

Without being bound by theory, the longer dissolution time of fumaric acid can be beneficial because, upon administration, fumaric acid does not dissolve as quickly as other acid solubilizers. Thus, fumaric acid can provide an acidic environment around the cenicriviroc or salt thereof for a longer period of time than other, more soluble acid solubilizers such as citric acid.

In addition to cenicriviroc and fumaric acid, the composition can have one or more additional ingredients, for example one or more fillers, one or more disintegrants, or one or more lubricants. Further additional ingredients can also be present, although it should be understood that no particular additional ingredient is required unless otherwise specified.

The one or more fillers, when used, can include at least one of microcrystalline cellulose, calcium phosphate dibasic, cellulose, lactose, sucrose, mannitol, sorbitol, starch, and calcium carbonate. For example, the one or more fillers can be microcrystalline cellulose. The weight ratio of the one or more fillers, such as microcrystalline cellulose, to the cenicriviroc or salt thereof can be from about 25:10 to about 10:8, such as from about 20:10 to about 10:10 or about 15:10, based on the weight of free cenicriviroc. The one or more fillers, such as microcrystalline cellulose, can be present in an amount of from about 25% to about 55%, such as from about 30% to about 50%, or about 40%, by weight of the composition.

The one or more disintegrants, when used, can include at least one of cross-linked polyvinylpyrrolidone, cross-linked sodium carboxymethyl cellulose, and sodium starch glycolate. For example, the one or more disintegrants can be cross-linked sodium carboxymethyl cellulose. The weight ratio of the one or more disintegrants, such as cross-linked sodium carboxymethyl cellulose, to the cenicriviroc or salt thereof can be from about 10:100 to about 30:100, such as about 25:100 based on the weight of free cenicriviroc. The one or more disintegrants can be present in an amount of from about 2% to about 10%, such as about 4% to about 8%, or about 6%, by weight of the composition.

The one or more lubricants, when used, can include at least one of talc, silica, stearin, magnesium stearate, or stearic acid. For example, the one or more lubricants can be magnesium stearate. The one or more lubricants can be present in an amount of from about 0.25% to about 5%, such as from about 0.75% to about 3%, or about 1.25%, by weight of the composition.

Further additional ingredients that can be used are listed in Remington: The Science and Practice of Pharmacy, which is hereby incorporated by reference in its entirety for all purposes.

The composition can be in various forms. Examples of forms suitable for pharmaceutical use are listed in Remington: The Science and Practice of Pharmacy, which is hereby incorporated by reference in its entirety for all purposes. The composition can be, for example, a granulate, a matrix, a tablet, or portion of a tablet, such as one or more layers of a multi-layer tablet. The composition can be a powder, which can be filled into a capsule, sachet, bottle, vial, ampoule, etc. The composition can be a substrate for a one or more coating layers, such as pharmaceutical coating layers known in the art, which can be applied to the composition. When the composition is a granulate, the average particle size can be about 75 microns or greater, such as about 300 microns or greater.

The composition can be manufactured by admixing the cenicriviroc or salt thereof, such as cenicriviroc mesylate, with fumaric acid to form an admixture and dry granulating the admixture. Exemplary methods of dry granulation include roller compaction, slugging, and pelletization. The size of the dry granulated composition can the be reduced by methods such as milling, if desired. However, it should be understood that no particular methods of granulation, dry granulation, or size reduction are required unless otherwise specified. One or more of the fillers, disintegrants, lubricants, and other additional ingredients discussed above can also be admixed in the admixture. The ratio or amounts of the various components of the admixture can be the same as those discussed above with reference to the composition. The dry granulated admixture can have an average particle size of greater than 75 microns, such as greater than 300 microns.

Dry granulation can produce a composition that not only has a low level of water, but also is not significantly hygroscopic, that is, does not absorb significant amounts of additional water from the surrounding environment. For example, the water content of the composition can be no more than about 4%, or no more than about 2%, by weight after about six weeks of exposure to about 40° C. at about 75% relative humidity when packaged with a desiccant.

After dry granulation, the composition can be formulated into one or more formulations. For example, the composition can be filled into a capsule or sachet. As further examples, the dry granulated admixture can be formulated into a matrix, a tablet, or one or more layers of a single or multi-layer tablet, for example by compression, or further formulated by methods known in the art for formulating pharmaceutical compositions, such as those described in Remington: The Science and Practice of Pharmacy, which is hereby incorporated by reference in its entirety for all purposes.

The composition, for example in the form of a granulate, can be mixed with other granulates or powders, however, such extragranular materials, which are not granulated with the components of the composition, are not part of the composition, for example, for purposes of calculating the ratio or relative amounts of the various components. However, one or more formulations comprising the composition in the form of a granulate and further comprising extragranular materials are contemplated as part of the embodiments described herein.

As an example, a formulation can include a composition as described herein in the form of granulate along with one or more extragranular components, such as one or more additional pharmaceutically active agents. The one or more additional pharmaceutically active agents can include one or more of antiretroviral drugs, such as one or more CCR5 receptor antagonists, entry inhibitors, nucleoside reverse transcriptase inhibitors, nucleotide reverse transcriptase inhibitors, non-nucleoside reverse transcriptase inhibitors, protease inhibitors, integrase inhibitors, and maturation inhibitors, for example, one or more of maraviroc, lamivudine, efavirenz, raltegravir, vivecon, bevirimat, alpha interferon, zidovudine, abacavir, lopinavir, ritonavir, tenofovir, tenofovir disoproxil, tenofovir prodrugs, emtricitabine, elvitegravir, cobicistat darunavir, atazanavir, rilpivirine, and dolutegravir. As another example, the one or more additional pharmaceutically active agents can include one or more immune system suppressing agents, such as one or more of cyclosporine, tacrolimus, prednisolone, hydrocortisone, sirolimus, everolimus, azathioprine, mycophenolic acid, methotrexate, basiliximab, daclizumab, rituximab, anti-thymocyte globulin, and anti-lymphocite globulin, for example, tacrolimus or methotrexate.

For example, a composition as described herein can be admixed with the one or more additional pharmaceutically active agents and optionally one or more excipients, and then compressed into a monolithic fixed-dose combination tablet. As another example, a composition as described herein and a second composition comprising an additional pharmaceutically active agent can be formed into a multi-layer tablet by the use of tabletting equipment known in the art to be suitable for that purpose.

Current treatment guidelines for HIV prefer fixed-dose combination (FDC) single tablets. The main advantage of FDC products is the convenience and simplicity of dosing, which leads to increased patient compliance and improved clinical outcomes. FDC products for the HIV treatment fall into three categories: (1) Backbone formulations where 2 agents are co-formulated in a single tablet, e.g. Truvada (emtricitabine/tenofovir disoproxil fumarate), and Epzicom (Abacavir/lamivudine); (2) Boosted protease single tablets products, such as Kaletra (lopinavir/ritonavir); (3) Single Tablet Regimen (STR) products containing a complete treatment regimen in a single tablet, taken once-daily such as Atripla (efavirenz/emtricitabine/tenofovir disoproxil fumarate), Complera (emtricitabine/rilpivirine/tenofovir disoproxil fumarate), and Stribild (elvitegravir/cobicistat/emtricitabine/tenofovir disoproxil fumarate).

In one embodiment, the invention provides a composition comprising cenicriviroc or a salt thereof and fumaric acid in combination with lamivudine (3TC). In another embodiment, the invention provides a composition comprising cenicriviroc or a salt thereof and fumaric acid in combination with efavirenz (EFV). In yet another embodiment, the invention provides a composition comprising cenicriviroc or a salt thereof and fumaric acid in combination with 3TC and EFV. In certain embodiments, the combination products containing cenicriviroc, 3TC and/or EFV prepared according to the invention are effective as single tablet regiment for the treatment of viral infection, in particular, HIV infection.

In one embodiment, the dose strength ratio of cenicriviroc to 3TC in combination formulations is from about 1:2 to about 1:12, such as about 1:2, 1:4, 1:10 or 1:12 based on the weight of free cenicriviroc, inclusive of all ranges and subranges therebetween. For example, a single tablet comprising cenicriviroc or its salt and 3TC may comprise a dose strength of 25 mg of cenicriviroc free base and 300 mg of 3TC thereby providing a dose strength ratio of 1:12. Alternatively, a single tablet comprising cenicriviroc or its salt and 3TC may comprise a dose strength of 150 mg of cenicriviroc free base and 300 mg of 3TC thereby providing a dose strength ratio of 1:2.

In one embodiment, the dose strength ratio of cenicriviroc to EFV in combination formulations is from about 1:2 to about 1:12, such as about 1:2, 1:3, 1:4, 1:5, 1:6, 1:8, 1:10 or 1:12 based on the weight of free cenicriviroc, inclusive of all ranges and subranges therebetween. For example, a single tablet comprising cenicriviroc or its salt and EFV may comprise a dose strength of 150 mg of cenicriviroc free base and 600 mg of EFV thereby providing a dose strength ratio of 1:4. Alternatively, a single tablet comprising cenicriviroc or its salt and EFV may comprise a dose strength of 120 mg of cenicriviroc free base and 600 mg of EFV thereby providing a dose strength ratio of 1:2.

The invention also provides methods of preparing combination formulations comprising cenicriviroc, 3TC and/or EFV. In one embodiment, the method of preparing combination formulations comprises admixing cenicriviroc or a salt thereof, fumaric acid, and other pharmaceutical excipients to form an admixture, dry granulating the admixture to obtain cenicriviroc granules, blending the cenicriviroc granules with 3TC and/or EFV and suitable excipients and compressing the resulting mixture into tablets to obtain a combination product. That is, in this embodiment, the additional active agents are present extragranularly. In alternative embodiments, a portion or the entire amount of additional active agents may be present intragranularly. In yet another embodiment, combination products comprising cenicriviroc, 3TC and EFV may be prepared in the form of a bilayer tablet where one layer comprises cenicriviroc and 3TC and the other layer comprises EFV. In one embodiment of the bilayer tablets, cenicriviroc is present intragranularly and 3TC is present extragranularly.

### EXAMPLES

### Example 1

A series of cenicriviroc mesylate compositions that were identical except for the identity of the acid solubilizer were prepared by wet granulation in a Key 1 L bowl granulator, followed by tray drying, sieving, mixing and compression into tablets on a Carver press. The composition of the formulations is shown in Table 2.

TABLE 2

| | Unit Formula (mg/unit) | | | |
|---|---|---|---|---|
| | Example | Example | Example | Example |
| | 1a | 1b | 1c | 1d |
| | Citric | Fumaric | Maleic | Sodium |
| Components | Acid | Acid | Acid | Bisulfate |
| Cenicriviroc Mesylate | 28.45 | 28.45 | 28.45 | 28.45 |
| Mannitol | 7.88 | 7.88 | 7.88 | 7.88 |
| Hydroxypropyl | 2.62 | 2.62 | 2.62 | 2.62 |
| Cellulose | | | | |
| Cross-linked sodium | 1.75 | 1.75 | 1.75 | 1.75 |
| carboxymethyl | | | | |
| cellulose | | | | |
| Citric Acid | 43.75 | — | — | — |
| Fumaric Acid | — | 43.75 | — | — |
| Maleic Acid | — | — | 43.75 | — |
| Sodium Bisulfate | — | — | — | 43.75 |
| Silicon Dioxide | 0.43 | 0.43 | 0.43 | 0.43 |
| Magnesium Stearate | 0.88 | 0.88 | 0.88 | 0.88 |
| Total | 87.5 | 87.5 | 87.5 | 87.5 |

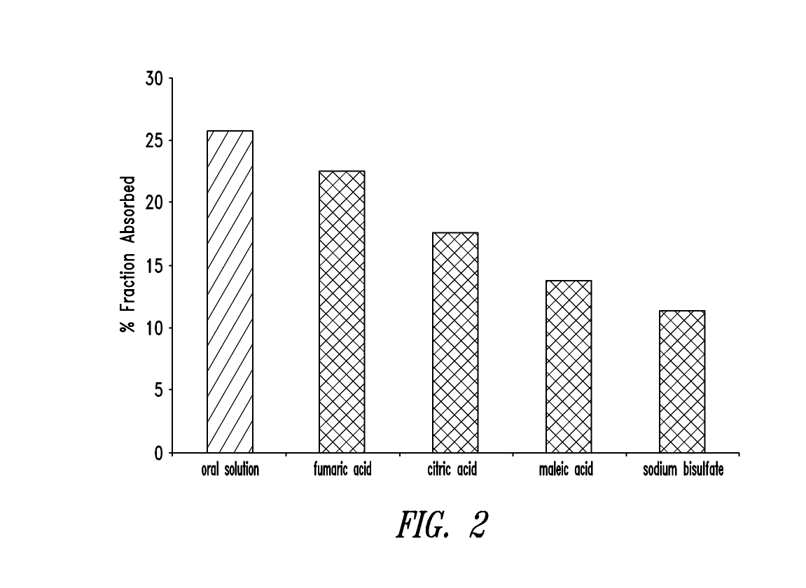

The tablets were administered to beagle dogs. An oral solution was also administered as a control. The absolute bioavailabilities of the formulations and of the oral solution were determined, and are shown in FIG. 2. The result shows that the cenicriviroc mesylate with fumaric acid has a significantly higher bioavailability than any of the other solubilizers tested.

### Examples 2a-2e

Cenicriviroc mesylate, fumaric acid, microcrystalline cellulose, cross-linked sodium carboxymethyl cellulose, cross-linked polyvinylpyrrolidone (when used), and magnesium stearate were admixed, dry granulated, milled, blended with extragranular microcrystalline cellulose, cross-linked sodium carboxymethyl cellulose, and magnesium stearate and compressed into tablets. In Example 2c, the fumaric acid was not granulated with the cenicriviroc mesylate and other excipients; instead, it was admixed with the extragranular microcrystalline cellulose, and this admixture blended with the dry granulate before compression into tablets. In Example 2a, 39.00 mg of the cross-linked sodium carboxymethyl cellulose was part of the dry granulate; the rest was admixed with the extragranular microcrystalline cellulose and this admixture blended with the dry granulate before compression into tablets. All of the tablets had a hardness greater than 10 kP and friability less than 0.8% w/w. The tablets had the compositions shown in Table 3a.

TABLE 3a

| | Unit Formula (mg/unit) | | | | |
|---|---|---|---|---|---|
| | Example | Example | Example | Example | Example |
| Components | 2a | 2b | 2c | 2d | 2e |
| Cenicriviroc Mesylate | 170.69^{a} | 170.69^{a} | 170.69^{a} | 170.69^{a} | 170.69^{a} |
| Fumaric Acid | 160.00 | 160.00 | 160.00^{b} | 160.00 | 80.00 |
| Microcrystalline | 252.68 | 272.18 | 272.18 | 272.18 | 66.35 |
| Cellulose | | | | | |
| Cross-linked | — | — | — | 19.50 | — |
| polyvinylpyrrolidone | | | | | |
| Cross-linked sodium | 58.50 | 39.00 | 39.00 | 19.50 | 20.70 |
| carboxymethyl | | | | | |
| cellulose | | | | | |
| Magnesium Stearate | 8.13 | 8.13 | 8.13 | 8.13 | 2.55 |
| Total | 650.0 | 650.0 | 650.0 | 650.0 | 340.0 |

^{a}Equivalent to 150 mg cenicriviroc freebase.
^{b}Added in the extragranular portion of the powder blend.

The concentration percentage (w/w) and mass per tablet of the components of Example 2b are shown in Table 3b.

TABLE 3b

| | Concentration | Mass (mg) per |
|---|---|---|
| Component | (% w/w) | tablet |
| Cenicriviroc mesylate | 26.26 | 170.69^{a} |
| Fumaric acid | 24.62 | 160.00 |
| Microcrystalline | 41.87 | 272.18 |
| cellulose | | |
| Cross-linked sodium | 6.00 | 39.00 |
| carboxymethyl cellulose | | |
| Magnesium stearate | 1.25 | 8.13 |
| Total | 100.0 | 650.0 |

^{a}equivalent to 150 mg cenicriviroc free base

### Example 3

Cenicriviroc mesylate, microcrystalline cellulose, cross-linked sodium carboxymethyl cellulose, and magnesium stearate were admixed, dry granulated, dried, milled, blended with extragranular microcrystalline cellulose, cross-linked sodium carboxymethyl cellulose, fumaric acid, colloidal silicon dioxide, and magnesium stearate and compressed into tablets having a hardness greater than 10 kP and friability less than 0.8% w/w. The resulting tablets had the composition shown in Table 4.

TABLE 4

| | Concentration | Mass (mg) per |
|---|---|---|
| Component | (% w/w) | tablet |
| Cenicriviroc mesylate | 26.26 | 28.45^{a} |
| Fumaric acid | 24.62 | 26.67 |
| Microcrystalline | 41.87 | 45.36 |
| cellulose | | |
| Cross-linked sodium | 6.00 | 39.00 |
| carboxymethyl | | |
| cellulose | | |
| Magnesium stearate | 1.25 | 1.35 |
| Total | 100.0 | 108.3 |

^{a}equivalent to 25 mg cenicriviroc free base

Notably, the formulation of Table 4 has the same ratio of components as that of Table 3b, and differs only in the total amount of the components that are used for each tablet. Thus, Table 3b shows tablets with 150 mg cenicriviroc (based on free base), whereas Table 4 shows tablets with 25 mg cenicriviroc (based on free base) with the same ratio of components as the 150 mg tablets of Example 2b, shown in Table 3b.

### Example 4

### Reference

The citric acid based formulation of Table 5 was prepared as follows. Cenicriviroc, hydroxypropyl cellulose, mannitol, and cross-linked sodium carboxymethyl cellulose were admixed, wet granulated, dried, milled, and blended with microcrystalline cellulose, cross-linked sodium carboxymethyl cellulose, citric acid, colloidal silicon dioxide, talc, and magnesium stearate. The resulting blend was compressed into tablets having a hardness greater than 10 kP and friability less than 0.8% w/w. The tablets were coated with hydroxypropyl methylcellulose, polyethylene glycol 8000, titanium dioxide, and yellow iron oxide. The coated tablets thus produced were substantially identical to those disclosed in U.S. Patent Application Publication No. 2008/031942 (see, e.g., Table 3).

TABLE 5

| Component | mg/tablet | % w/w |
|---|---|---|
| Cenicriviroc mesylate | 28.91 | 4.68 |
| Mannitol | 341.09 | 56.85 |
| Microcrystalline | 80.00 | 12.94 |
| cellulose | | |
| Colloidal silicon dioxide | 12.00 | 2.00 |
| Citric acid anhydrous | 75.00 | 12.14 |
| Hydroxypropyl cellulose | 12.00 | 1.94 |
| Cross-linked sodium | 30.00 | 4.85 |
| carboxymethyl | | |
| cellulose | | |
| Talc | 12.00 | 1.94 |
| Magnesium stearate | 9.00 | 1.46 |
| Hydroxypropyl | 11.71 | 1.89 |
| methylcellulose | | |
| Polyethylene glycol 8000 | 2.69 | 0.44 |
| Titanium dioxide | 3.03 | 0.49 |
| Yellow iron oxide | 0.57 | 0.09 |

### Example 5

### Reference

### Example 5a

Cenicriviroc and hydroxypropyl methylcellulose acetate succinate were dissolved in methanol and spray dried into a fine powder containing 25% cenicriviroc by weight (based on the weight of cenicriviroc free base). The powder was admixed with colloidal silicon dioxide, microcrystalline cellulose, mannitol, sodium lauryl sulfate, cross-linked sodium carboxymethyl cellulose, and magnesium stearate. The admixture was compressed into tablets having a hardness greater than 10 kP and friability less than 0.8% w/w. The final composition of the tablets is shown in Table 6a.

TABLE 6a

| Component | Weight % | Mass (mg) |
|---|---|---|
| Cenicriviroc (as | 8.33 | 50.00 |
| mesylate salt) | | |
| Hydroxypropyl | 25.00 | 150.00 |
| methylcellulose acetate | | |
| succinate | | |
| Sodium lauryl sulfate | 2.00 | 12.00 |
| Cross-linked sodium | 6.00 | 36.00 |
| carboxymethyl | | |
| cellulose | | |
| Microcrystalline | 27.83 | 167.00 |
| cellulose | | |
| Mannitol | 27.83 | 167.00 |
| Colloidal silicon dioxide | 1.00 | 6.00 |
| Magnesium stearate | 2.00 | 12.00 |
| Total | 100.0 | 600.0 |

### Example 5b

### Film-Coated Composition of Example 5a

Cenicriviroc and hypromellose acetate succinate were dissolved in methanol and spray dried into a fine powder containing 25% CVC parent by weight. The powder was admixed with colloidal silicon dioxide, microcrystalline cellulose, mannitol, sodium lauryl sulfate, cross-linked sodium carboxymethyl cellulose, and magnesium stearate. The admixture was compressed into tablets having a hardness greater than 10 kp and friability less than 0.8% w/w. The tablets were then film-coated with Opadry Yellow 21K120001 (Colorcon) to a theoretical weight gain of 3.5%. The final composition of the tablets is shown in Table 6b.

TABLE 6b

| | Concentration | Mass (mg) per |
|---|---|---|
| Components | (% w/w) | tablet |
| Cenicriviroc (as the | 8.33 | 50.00 |
| mesylate salt) | | |
| Hypromellose acetate | 25.00 | 150.00 |
| succinate | | |
| Sodium lauryl sulfate | 2.00 | 12.00 |
| Cross-linked sodium | 6.00 | 36.00 |
| carboxymethyl | | |
| cellulose | | |
| Microcrystalline | 27.83 | 167.00 |
| cellulose | | |
| Mannitol | 27.83 | 167.00 |
| Colloidal silicon dioxide | 1.00 | 6.00 |
| Magnesium stearate | 2.00 | 12.00 |
| Total | 100.0 | 600.0^{a} |
| Opadry Yellow 21K120001^{b} | 3.5^{c} | 21.0^{c} |

^{a}Tablet weight is adjusted to accommodate the increase in weight for the adjustment of purity and the mesylate salt correction factor.
^{b}Opadry II Yellow 21K12001 (Colorcon) contains ethylcellulose; hypromellose, USP; triacetin; titanium dioxide, USP; yellow iron oxide.
^{c}Film-coat weight is a theoretical weight gain of 3.5% w/w on the tablet core.

### Example 6

The absolute bioavailability of the tablets of Example 3 in beagle dogs was compared to that of the tablets of Examples 4 and 5, as well as to both an oral solution of cenicriviroc mesylate and a gelatin capsule containing cenicriviroc mesylate powder. The results are shown in Table 7.

TABLE 7

| Component | Absolute bioavailability(%) |
|---|---|
| Oral Solution | 25.8 |
| Powder in capsule | 6.4 |
| Example 3 | 26.6 |
| Example 4 | 21.1 |
| Example 5 | 12.4 |

This example demonstrates that the bioavailability of cenicriviroc in dry granulated tablets with fumaric acid (Example 3) is substantially similar to that of an oral solution, and is significantly higher than the bioavailability of cenicriviroc in wet granulated tablets with citric acid (Example 4), and over double that of cenicriviroc in tablets with amorphous cenicriviroc in a spray dried dispersion with HPMC-AS (Example 5). These results are surprising, because there was no reason to suspect that dry granulation of crystalline API provides a significant increase in bioavailability over wet granulation and amorphous spray dried dispersions. This is especially so because amorphous spray dried dispersions are frequently used to increase the bioavailability of poorly water soluble drugs. These results are also surprising because fumaric acid has a slower dissolution time than citric acid and was used at a lower mass ratio of acid relative to cenicriviroc API (3:1 for citric acid:API versus 1.06:1 fumaric acid:API). Thus, the finding that fumaric acid is a more effective solubilizer for cenicriviroc than citric acid is surprising and unexpected.

### Example 7

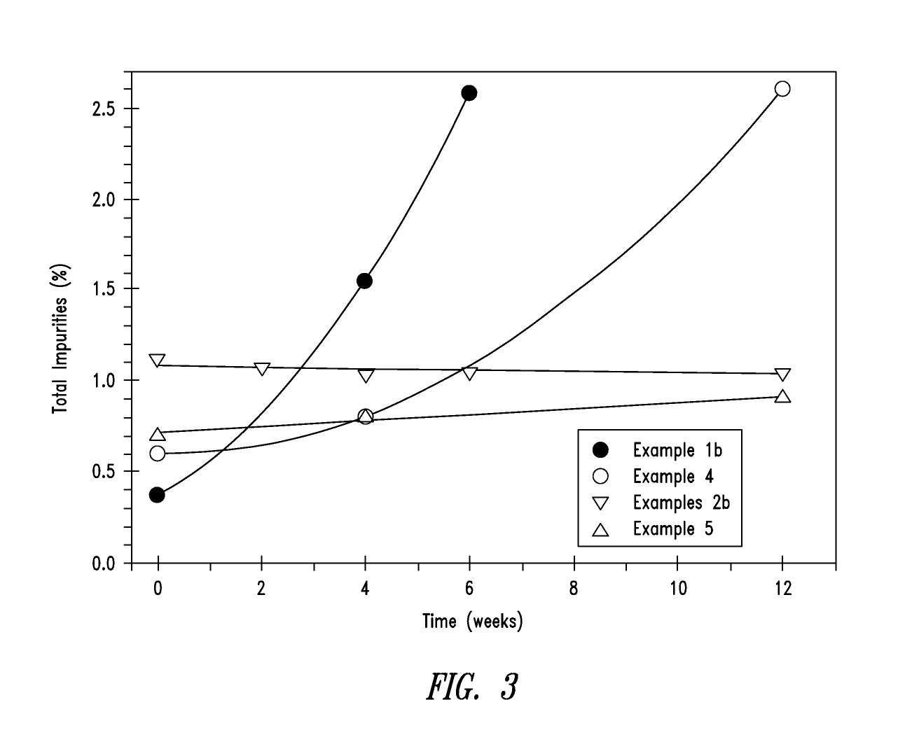

The stability under an accelerated stability test of the tablets of Example 2b was compared to that of the tablets of Examples 1b, 4, and 5 by exposing tablets of each of those Examples to an environment of 75% relative humidity at 40° C. All tablets were packaged with a desiccant in an induction sealed bottle during the study. As shown in FIG. 3, the tablets of Examples 2b are surprisingly much more stable than the other wet granulated tablets, and have a stability similar to that of the spray dried dispersion tablets. This difference in stability between the tablets of Examples 2b and Example 4 is particularly surprising since the only significant difference between the two is the method of making the formulations (dry granulation vs. wet granulation). These results are also surprising, because it was not previously known that the method of granulation could have an effect on both cenicriviroc bioavailability and tablet stability.

### Example 8

The stability under an accelerated stability test of the tablet of Example 2b was tested by exposing the tablets to an environment of 75% relative humidity at 40° C. for six weeks. All tablets were packaged with a desiccant in an induction sealed bottle during the study. The tablets were tested for water content, strength, and total impurities. The results are shown in Table 8, which shows that the tablets are very stable under these conditions.

TABLE 8

| Time | Water content | Strength | Total Impurities |
|---|---|---|---|
| (Weeks) | (%) | (%) | (%) |
| 0 | 1.5 | 99.1 | 1.2 |
| 2 | 1.4 | 99.2 | 1.1 |
| 4 | 1.4 | 98.0 | 1.0 |
| 6 | 1.4 | 98.6 | 1.0 |

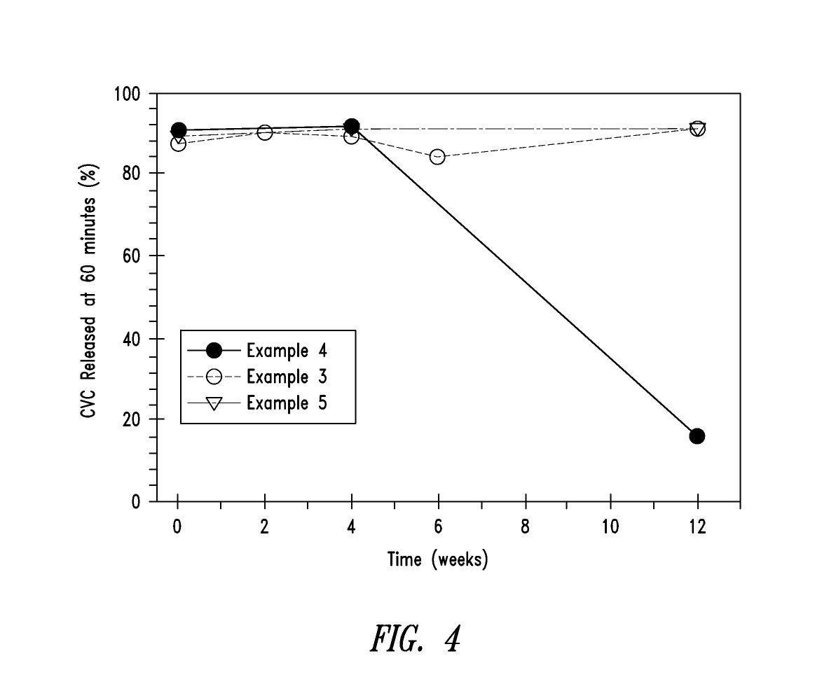

The dissolution profile of cenicriviroc from tablets of Examples 3, 4, and 5 were also tested after storage under the conditions described above. The results appear in FIG. 4, which shows that the wet-granulated citric acid containing tablet of Example 4 was much less stable than the dry granulated fumaric acid containing tablet of Example 3 and the spray-dried dispersion tablet of Example 5.

### Example 9

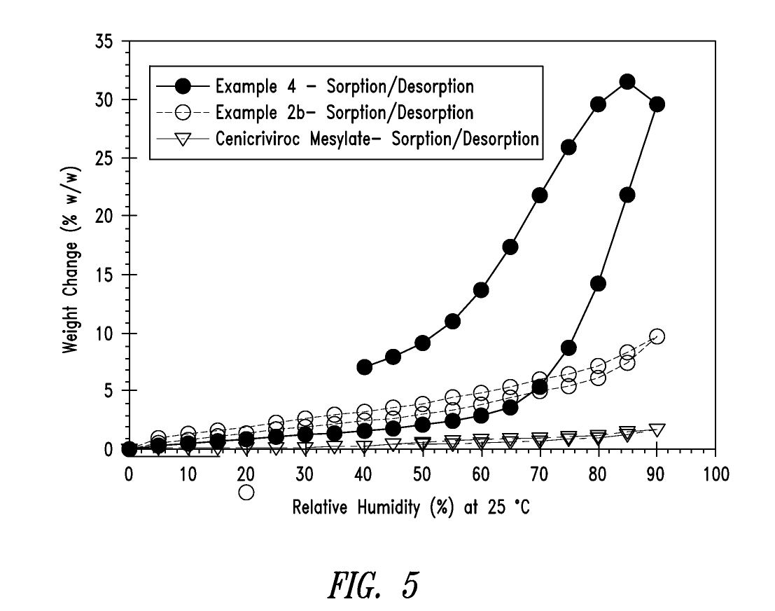

Dynamic vapor sorption isotherms at 25° C. correlate to the stability of the tablets of Examples 2b and 4 with that of cenicriviroc mesylate. Sorption was performed from 0% relative humidity to 90% relative humidity at 5% intervals. At each interval, each sample was equilibrated for no less than 10 minutes and no longer than 30 minutes. Equilibration was stopped when the rate of mass increase was no more than 0.03% w/w per minute or after 30 minutes, whichever was shorter. The result, which appears in FIG. 5, shows that tablets of Example 2b are significantly more stable than those of Example 4. This result is consistent with Example 2b being significantly less hygroscopic than Example 4. The increased hygroscopicity of Example 4, in comparison to Examples 2b, can be associated with a higher mobile water content which can in turn cause partial gelation and subsequent decreased stability of Example 4.

### Example 10

The bioavailability of the tablets of Example 3 was compared to that of Example 5 and cenicriviroc mesylate powder in a gelatin capsule in different stomach states in beagle dogs (n=5). The bioavailability was tested under different pre-treatment states, each of which alters the gastric pH. Specifically, pentagastric pretreatment provides the lowest pH, no treatment provides an intermediate pH, and famotidine treatment provides the highest pH. Pentagastrin is a synthetic polypeptide that stimulates the production of gastric acid thereby lowering the gastric pH.

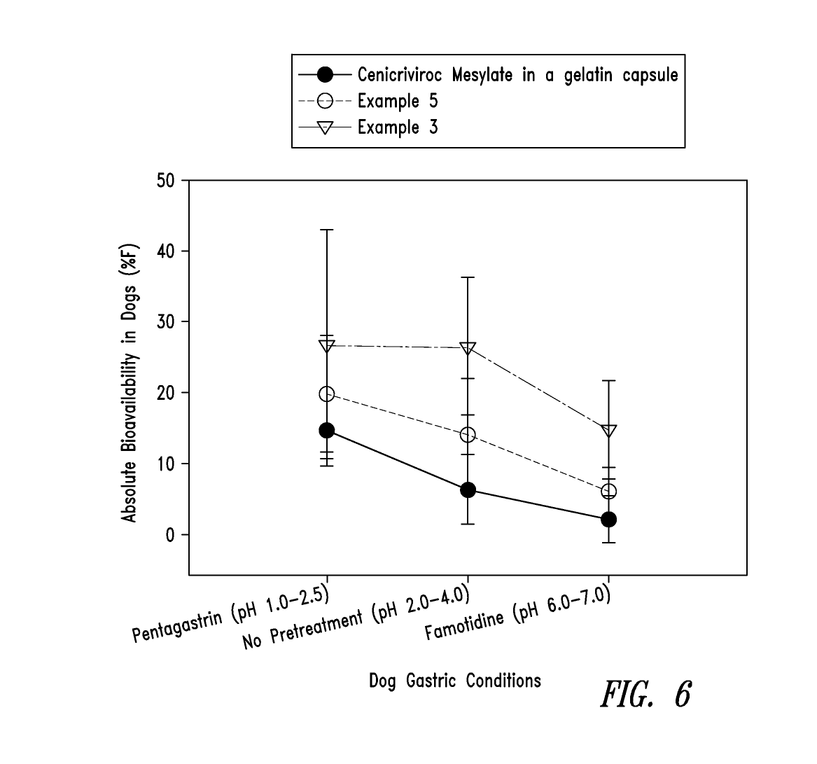

The result, which appears in FIG. 6, shows that the tablets of Example 3 has a higher bioavailability under all conditions that were tested. The bioavailability of Example 3 varied less between pentagastrin treated and untreated dogs, whereas Example 5 showed a significant loss of bioavailability in fasted, non-treated dogs (intermediate gastric pH) compared to that in pentagastrin treated dogs (lowest gastric pH). Pretreatment with famotidine, an H2 receptor agonist that suppresses stomach acidity and raises gastric pH decreased bioavailability for all samples, however, the reduction for Example 3 was much less than that for Example 5.

These results demonstrate an additional unexpected benefit of dry granulated cenicriviroc compositions with fumaric acid. Specifically, the pharmacokinetics of such formulations do not vary as much as those of the spray dried dispersion formulation of Example 5 when administered across the full range of potential human gastric pH conditions. This result is unexpected and surprising, because the bioavailability of other weakly basic antiretroviral drugs, such as atazanavir, is greatly effected by the gastric pH. For such drugs, changes in gastric pH, which can be caused by a disease or medical condition, such as achlorohydric patients, or by co-administration of drugs such as antacids, proton pump inhibitors, or H2 receptor agonists, can lower the bioavailability to sub-therapeutic levels. These results showing that the dry granulated, fumaric acid based cenicriviroc mesylate formulation of Example 3 is less prone to bioavailability changes as the gastric pH changes shows that Example 3 is a more robust formulation that can be used in patients who have or are likely to have varying gastric pH levels.

### Example 11

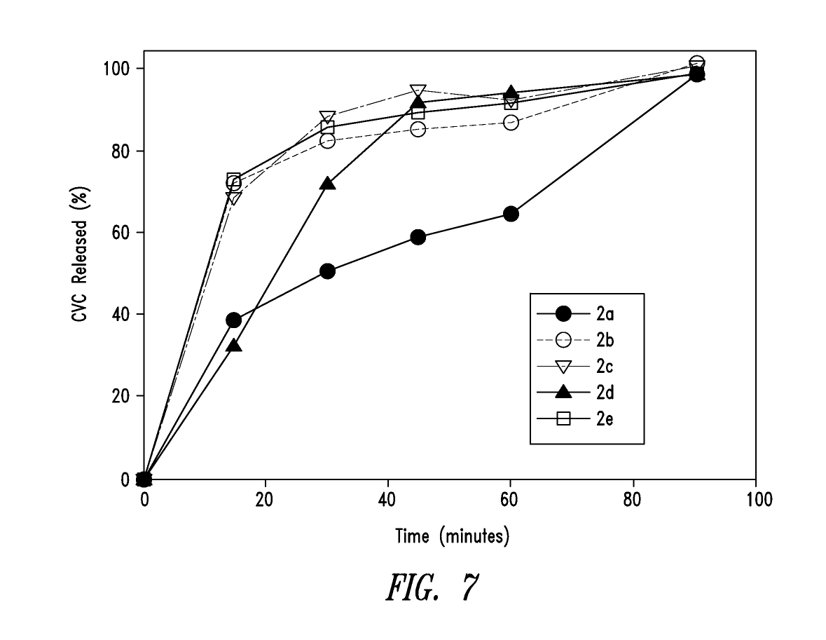

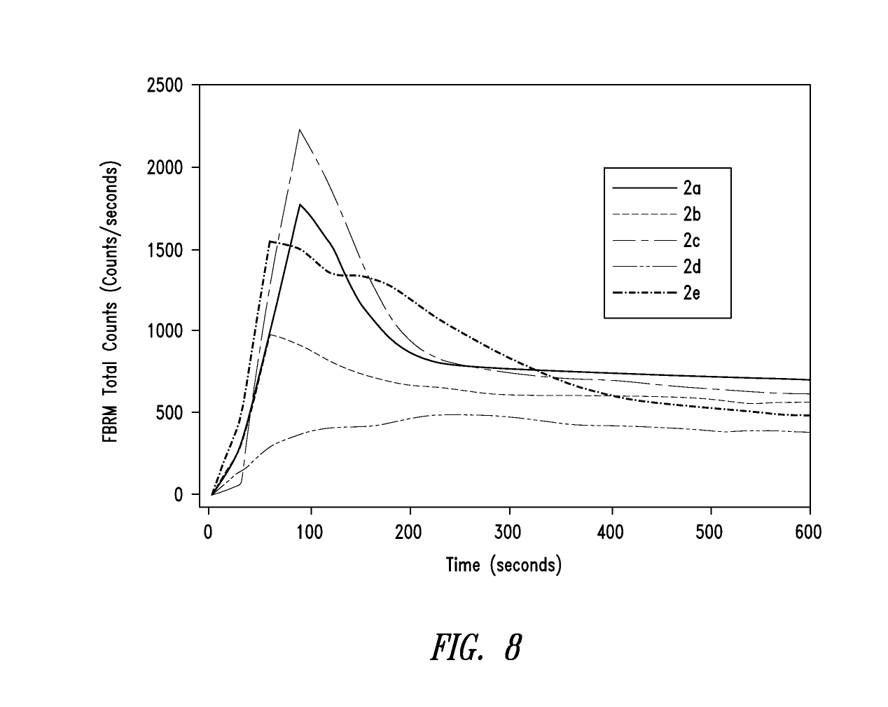

The dissolution profile of the formulations of Examples 2a-2e were measured using a USP Type 2 apparatus at 50 rpm paddle speed in 0.1 N HCl with 0.1% (w/w) CTAB. The results are shown in FIG. 7. The disintegration profiles of the formulations of Examples 2a-2e were measured using FBRM. These results are shown in FIG. 8. Together, FIGS. 7 and 8 show that compositions and formulations containing cenicriviroc mesylate and fumaric acid having different dissolution profiles can be obtained.

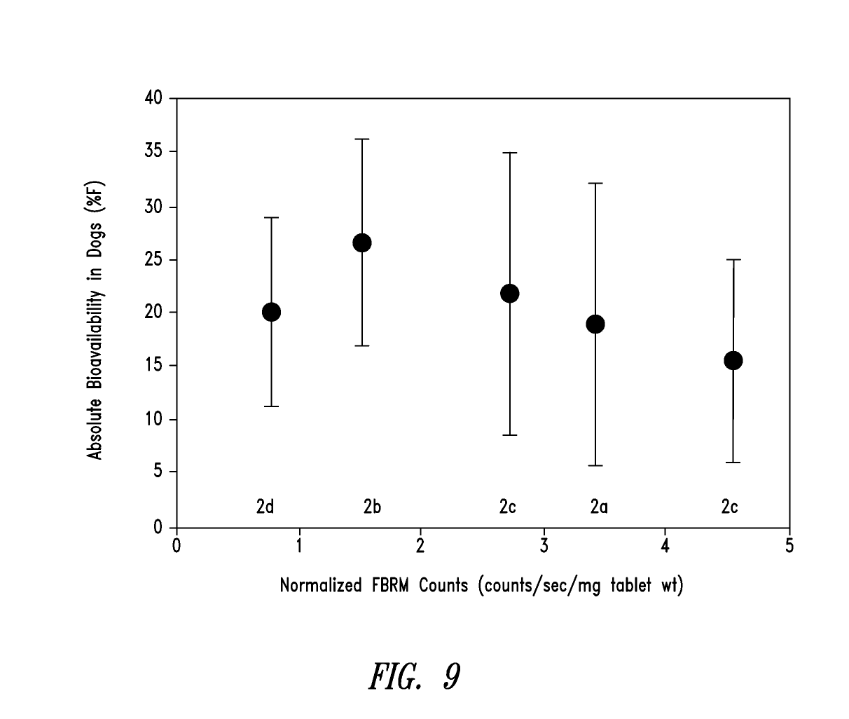

The absolute bioavailability in beagle dogs (n=5) of samples 2a-2e was also obtained, and the results are shown in FIG. 9. The results show that while the absolute bioavailability may vary depending on the formulation, a high bioavailability was obtained for all samples.

### Example 12

In this study, tablets of Example 2 were coated with commercially available film-coating formulations and the stability of film-coated tablets was tested under accelerated conditions (40° C./75% RH).

A film-coating step is commonly employed for the purposes of taste masking or establishing a unique trade dress for the intended commercial formulation. Tablets of Example 2 were coated with three film-coating formulations, each formulation containing a different base polymer system. Specifically, Opadry II White 57U18539 containing hydroxy propyl methylcellulose (HPMC or hypromellose), Opadry II White 85F18422 (Colorcon) containing polyethylene glycol (PEG) and partially hydrolyzed polyvinyl alcohol (PVA), and Opadry II White 200F280000 containing a methyacrylic acid copolymer were used to coat the tablets.

Tablets were coated by atomizing an aqueous suspension of the coating formulation onto the tablet surface in a perforated coating pan. The pan was continuously circulated with warm processing air that provides convective heat transfer to evaporate water from the tablet surface, leaving the coating formulation deposited as a film layer on the tablet surface. Tablet compositions coated with the above-mentioned polymers are shown in Tables 9-11 below. Analysis of the surface of the film-coated tablets is summarized in Table 12.

### Example 12a

TABLE 9
(HPMC-coated CVC Single Agent)

| | Concentration | Mass (mg) per |
|---|---|---|
| Components | (% w/w) | tablet |
| Cenicriviroc Mesylate | 26.26 | 170.69^{a} |
| Fumaric Acid | 24.62 | 160.00 |
| Microcrystalline | 41.87 | 272.18 |
| Cellulose | | |
| Cross-linked Sodium | 6.00 | 39.00 |
| Carboxymethyl | | |
| Cellulose | | |
| Magnesium Stearate | 1.25 | 8.13 |
| Total | 100.0 | 650.0 |
| Opadry II White 57U18539^{b} | 4.0^{c} | 26.0^{c} |

^{a}Equivalent to 150 mg cenicriviroc freebase.
^{b}Opadry II White 57U18539 contains hypromellose, USP; maltodextrin, NF; medium chain triglycerides, NF; polydextrose, NF; talc, USP; titanium dioxide, USP.
^{c}Film-coat weight is a theoretical weight gain of 4.0% w/w on the tablet core.

### Example 12b

TABLE 10
(PEG/PVA-coated CVC Single Agent)

| | Concentration | Mass (mg) per |
|---|---|---|
| Components | (% w/w) | tablet |
| Cenicriviroc Mesylate | 26.26 | 170.69^{a} |
| Fumaric Acid | 24.62 | 160.00 |
| Microcrystalline | 41.87 | 272.18 |
| Cellulose | | |
| Cross-linked Sodium | 6.00 | 39.00 |
| Carboxymethyl | | |
| Cellulose | | |
| Magnesium Stearate | 1.25 | 8.13 |
| Total | 100.0 | 650.0 |
| Opadry II White 85F18422^{b} | 4.0^{c} | 26.0^{c} |

^{a}Equivalent to 150 mg cenicriviroc freebase.
^{b}Opadry II White 85F18422 (Colorcon) contains polyethylene glycol 3350, NF; polyvinyl alcohol, partially hydrolyzed, USP; talc, USP; titanium dioxide, USP.
^{c}Film-coat weight is a theoretical weight gain of 4.0% w/w on the tablet core.

### Example 12c

TABLE 11
(Methacrylate-coated CVC Single Agent)

| | Concentration | Mass (mg) per |
|---|---|---|
| Components | (% w/w) | tablet |
| Cenicriviroc Mesylate | 26.26 | 170.69^{a} |
| Fumaric Acid | 24.62 | 160.00 |
| Microcrystalline | 41.87 | 272.18 |
| Cellulose | | |
| Cross-linked Sodium | 6.00 | 39.00 |
| Carboxymethyl | | |
| Cellulose | | |
| Magnesium Stearate | 1.25 | 8.13 |
| Total | 100.0 | 650.0 |
| Opadry II White | 4.0^{c} | 26.0^{c} |
| 200F280000^{b} | | |

^{a}Equivalent to 150 mg cenicriviroc freebase.
^{b}Opadry II White 200F280000 (Colorcon) contains methyacrylic acid copolymer type C, USP; polyethylene glycol 3350, NF; polyvinyl alcohol, partially hydrolyzed, USP; sodium bicarbonate, USP; talc, USP; titanium dioxide, USP.
^{c}Film-coat weight is a theoretical weight gain of 4.0% w/w on the tablet core.

Analysis of the surface of the film-coated tablets is summarized in Table 12 below. Since the coating with Opadry II White 200F28000 (tablets of Example 12c, Table 11) did not show uniform coverage, the tablets of Example 12c were not tested for stability. The coatings of Examples 12a and 12b showed acceptable coverage and good adhesion to the tablet surface.

TABLE 12
Surface Analysis of the film-coatings

| Sample | Film-coating analysis |
|---|---|
| Example 1 | Smooth, uniform film-coat; complete coverage |
| Example 2 | Smooth, uniform film-coat; complete coverage |
| Example 3 | Incomplete film-coat coverage; evidence of |
| | film-coat picking; surface defects; yellow |
| | tablet core showing through defects (due to |
| | yellow color of CVC active ingredient) |

The stability of the film-coated tablets of Examples 12a and 12b were compared to that of the uncoated tablets of Example 2 after exposure to an environment of 75% relative humidity at 40° C. All tablets were packaged with a desiccant in an induction sealed bottle during the study. The results of the stability testing are shown in Table 13.

TABLE 13

| | Example 2 | Example 12a | Example 12b |
|---|---|---|---|
| | (uncoated) | (coated) | (coated) |
| | Total CVC | Total CVC | Total CVC |
| Time | Impurities | Impurities | Impurities |
| (Weeks) | (%) | (%) | (%) |
| 0 | 1.2 | 1.0 | 1.0 |
| 2 | 1.1 | ND | ND |
| 4 | ND | 1.0 | 1.3 |
| 6 | 1.0 | ND | ND |

N/D—not determined

As shown in Table 13, the tablets of Examples 12a and 12b showed acceptable stability profile similar to that of the uncoated tablets of Example 2 with no substantial formation of impurities or degradants. These results are promising because previous experiments have shown that processing of cenicriviroc tablets in the presence of aqueous environment had deleterious effects on the chemical and physical stability of the tablets.

### Example 13

In this study, the pharmacokinetic (PK) profiles of compositions of Example 2b (shown in Table 3b), Example 3 (shown in Table 4), and Example 5b (shown in Table 6b) were evaluated in human clinical trials. The composition of Example 5b was used as a reference.

A phase 2b proof of concept study (“Study 202”) was carried out using the composition of Example 5b to establish the PK profile for the 200 mg recommended cenicriviroc dose taken with breakfast. In Study 202, the patients were administered 200 mg dose of the composition of Example 5b once per day for 10 consecutive days. Since the formulation of Example 5b is a 50 mg tablet, the patients were required to take 4 tablets each time to administer the 200 mg dose.

In Study 110, a multiple dose regimen for the composition of Example 2b was evaluated. In this study, the patients were administered 150 mg dose of the composition of Example 2b with breakfast once per day for 10 consecutive days. Each time, the patients consumed a single tablet of the composition of Example 2b containing the 150 mg dose.

In Study 111, the PK profile of a 200 mg single dose regimen administered on an empty stomach just prior to or at bedtime was evaluated. The 200 mg dose was administered by consuming one tablet of Example 2b (150 mg dose) and two tablets of Example 3 (25 mg dose/tablet). The administration of three tablets to provide the 200 mg dose was solely based on the availability of the tablets of Examples 2b and 3 and not due to any limitations on making a 200 mg tablet of cenicriviroc according to the invention.

The PK profile obtained in the above studies is summarized in Table 14 below.

TABLE 14

| | Study 202 | | | | | |
|---|---|---|---|---|---|---|
| | Example 5b | | Study 110 | | Study 111 | |
| | 200 mg | | Example 2b | | Example 2b & 3 | |
| | CVC (DP6) | | 150 mg | | 200 mg | |
| Param- | Multiple Dose ^{a} | | CVC (DP7) | | CVC (DP7) | |
| eter | (Reference) | | Multiple Dose | | Single Dose | |
| AUC₀₋ₗₐₛₜ | 5274 | (2369) | 8568 | (3491) | 13732 | (3418) |
| Cmax | 406 | (181) | 620 | (220) | 624 | (159) |

| | | | | | |
|---|---|---|---|---|---|
| Cmin | 103 | (59) | 174 | (77) | — |

^{a} DP6 at 200 mg taken with breakfast achieves the exposure for efficacious clinical use of CVC in HIV-1 treatment infection based on Phase 2b data.

The above data shows that the AUC values obtained in Study 110 where the inventive composition was administered were 1.6-fold higher than the AUC values obtained in Study 202 where the reference composition was administered. Thus, under the steady state conditions (characterized as multiple dose exposure over 10 days), the administration of 150 mg of cenicriviroc in the form of inventive compositions with breakfast resulted in higher bioavailability of cenicriviroc than the administration of 200 mg of cenicriviroc in the form of a reference composition with breakfast. This data demonstrates that the inventive CVC compositions where the microenvironment comprises an acid and is thus pH-adjusted have superior bioavailability than the spray-dried dispersion formulation. Thus, the inventive compositions make it possible to use lower amounts of CVC per patient per day thereby reducing the cost of the medication. The use of lower amounts of CVC also reduces the tablet size and improves the ease of swallowing. The need for lower amounts of CVC also makes it possible to combine CVC with other antiretroviral agents in a single tablet.

Study 111 was conducted to evaluate the PK parameters upon administration of the inventive compositions at or immediately prior to bedtime. For the treatment of HIV, a combination of two or more active agents is preferred over a single active agent. For example, efavirenz (EFV) and lamivudine (3TC) are used in combination with each other or other active agents for the treatment of HIV. It is recommended that EFV-containing compositions be taken on an empty stomach preferably at or around bedtime. This is because the PK profile of EFV is influenced by food contents of the stomach and the administration of EFV is associated with side effects such as CNS toxicities (e.g. dizziness) which are mostly experienced around the time of highest plasma concentrations (Tmax). Bedtime dosing is preferred for managing these aspects of EFV administration. If cenicriviroc is to be co-administered or co-formulated with EFV, it is important that the administration of cenicriviroc achieves a desired exposure level when taken on an empty stomach at bedtime. Furthermore, EFV is a metabolic inducer of P450 (specifically the CYP3A4 enzyme). Higher activity of CYP3A4 leads to rapid metabolism of CVC and consequently lower absorption of CVC. Therefore, if cenicriviroc is to be administered in combination with EFV on an empty stomach around bedtime, it was estimated that higher amounts of CVC would be necessary to provide higher exposure levels to compensate for the metabolic effects of EFV on CVC.

The recommended cenicriviroc exposure level for the treatment of HIV has been established in Study 202 (see Table 14) using a reference formulation containing 200 mg of cenicriviroc in the form of spray-dried dispersion administered with breakfast. Various other clinical trials based on different formulations of cenicriviroc have established that the steady-state exposure level (AUC) of cenicriviroc (characterized as day 10 exposure) is approximately 1.5 fold higher than the exposure levels obtained from a single dose due to a long half-life of CVC which takes more than one dosing interval to accumulate up to steady-state levels. The expectation that higher amounts of CVC will be required for its combination with EFV was consistent with the above data on the steady-state and single-dose exposure levels.

Unexpectedly, Study 111 showed that a dosing of 200 mg of cenicriviroc in the form of inventive compositions around bedtime on an empty stomach achieved single-dose exposure levels that were 2.6 fold higher than the reference steady-state exposure levels (Table 14). That is, a single 200 mg dose of the inventive composition around bedtime had higher bioavailability than multiple 200 mg doses of the reference composition administered with breakfast. The CVC exposure levels achieved in Study 111 using a 200 mg dose of inventive compositions were more than sufficient to counteract the EFV metabolic effects or food effects. It was, therefore, concluded from Study 111 that lower than 200 mg of CVC would be optimal for its co-formulation with EFV in single tablet regimen (STR) products such as CVC/EFV/3TC. Accordingly, further studies on combination product prototyping used 150 mg of CVC for STR products containing CVC/EFV/3TC.

### Example 14

A dry-granulated CVC composition was prepared using a custom-made lab-scale roller compactor machine with smooth stainless steel counter-rotating rolls (25 mm diameter, 125 mm width, and 0.5 to 3 mm gap width). Spunbonded olefin (Tyvek®) sleeves were used to contain the powders pre- and post-roller compaction, providing adequate conveying of small powder quantities through the compaction zone.

Cenicriviroc mesylate, fumaric acid, microcrystalline cellulose, and cross-linked sodium carboxymethyl cellulose were admixed in a suitable-sized container and blended by a tumbling action for a total of 40 revolutions over 2 minutes. Magnesium stearate was added and the mixture was again blended for 40 revolutions over 2 minutes. To a Tyvek sheet of 100 mm×480 mm dimensions, a fold was made to form a sleeve that was 50 mm in width for a defined compaction zone that would contain the blended powder as it passed through the lab-scale roller compaction machine. Approximately 10 to 15 g of powder was added to the sleeve and distributed evenly. The powder-containing sleeve was fed to the roller compactor at a gap width of approximately 2 mm and with a speed of 45 rpm (linear velocity=0.06 m/s). The resulting ribbons were compacted to approximately 1.0 to 1.5 mm thickness measured using a digital caliper gauge. This process was repeated with more blended powder until the entire batch had been passed through the roller compactor completely. The resulting compacted ribbons were then milled to make granules using a 6 inch diameter, 20-mesh stainless steel rotary screen mill. The granules had the composition shown in Table 15.

TABLE 15

| | Concentration |
|---|---|
| Components | (% w/w) |
| Cenicriviroc Mesylate | 32.2 |
| Fumaric Acid | 30.2 |
| Microcrystalline | 33.0 |
| Cellulose | |
| Cross-linked Sodium | 3.7 |
| Carboxymethyl | |
| Cellulose | |
| Magnesium Stearate | 0.9 |
| Total | 100.0 |

The granules prepared above were further blended with microcrystalline cellulose, cross-linked sodium carboxymethyl cellulose, and magnesium stearate to prepare a CVC single agent tablet formulation shown in Table 16. The strength of the single agent tablet can be varied readily by simply adjusting the total tablet weight accordingly. For example, a tablet of 325 mg total mass could be prepared by simply using half the amounts of the components and would have 75 mg CVC freebase equivalent strength (linear scaling using a common blend), while maintaining the same ratio between the components as that in Table 16.

TABLE 16

| | Concentration | Mass (mg) per |
|---|---|---|
| Components | (% w/w) | tablet |
| Cenicriviroc Mesylate | 26.26 | 170.69^{a} |
| Fumaric Acid | 24.62 | 160.00 |
| Microcrystalline | 41.87 | 272.18 |
| Cellulose | | |
| Cross-linked Sodium | 6.00 | 39.00 |
| Carboxymethyl | | |
| Cellulose | | |
| Magnesium Stearate | 1.25 | 8.13 |
| Total | 100.0 | 650.0 |

^{a}Equivalent to 150 mg cenicriviroc freebase.

### Example 15

A single agent CVC tablet formulation containing lower excipient levels and thereby lower total tablet mass was prepared using the process described in Example 14. The tablets had the composition shown in Table 17. This formulation contains a higher concentration of cenicriviroc for the purposes of combining with other antiretroviral agents and to avoid overly large total tablet size for the combination product.

TABLE 17

| | Concentration |
|---|---|
| Components | (% w/w) |
| Cenicriviroc Mesylate | 40.5 |
| Fumaric Acid | 37.9 |
| Microcrystalline | 15.6 |
| Cellulose | |
| Cross-linked Sodium | 5.0 |
| Carboxymethyl | |
| Cellulose | |
| Magnesium Stearate | 1.0 |
| Total | 100.0 |

### Example 16

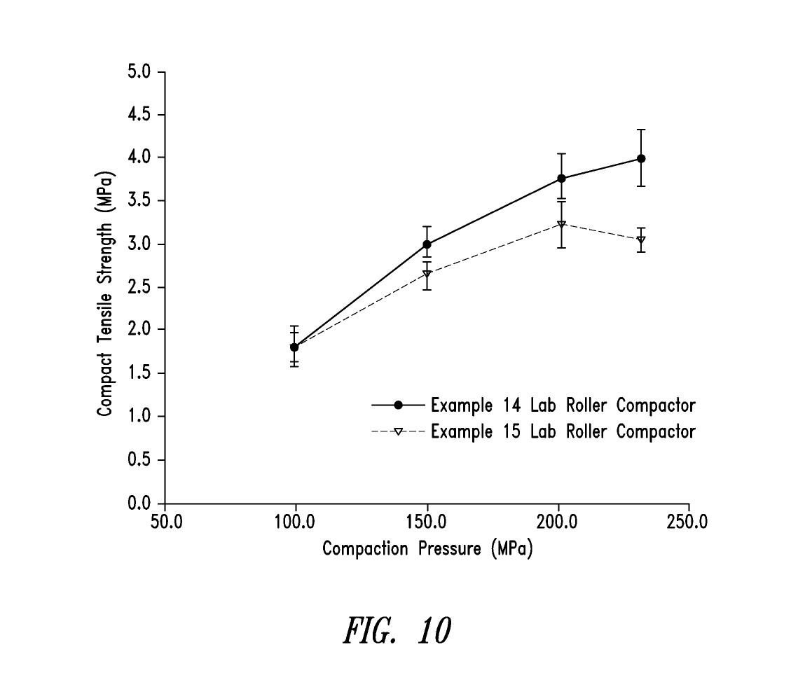

The compressibility of the milled granules prepared by the lab-scale roller compactor in Examples 14 and 15 was measured using the standard compressibility test and is shown in FIG. 10. Specifically, compression profiles of tablet blends were generated using an instrumented compaction device (Texture Analyzer) with ¼″ flat faced B tooling. Three replicates of 100 mg compacts were compressed at four forces ranging from 100 kg to 700 kg. The ejected compacts were immediately weighed on a four place balance and compact thickness was measured with precision calipers. Compacts were tested by diametric compression test to induce tensile failure. Tensile strength (TS) of the compacts is determined by the following equation:

TS=2·F/(π·D·T)

where F is the force needed to produce a tensile failure in the compact, D is the diameter of the compact, and T is the compact thickness. Solid fraction (SF) of the compact is calculated by the following equation:

SF=m/(V·ρ_{absolute})=m/[(π·(D/2)²·T)·ρ_{absolute}]

where m is the mass of the compact, V is the tablet volume, and ρabsolute is the absolute density of the tablet blend as measured with a helium pycnometer.

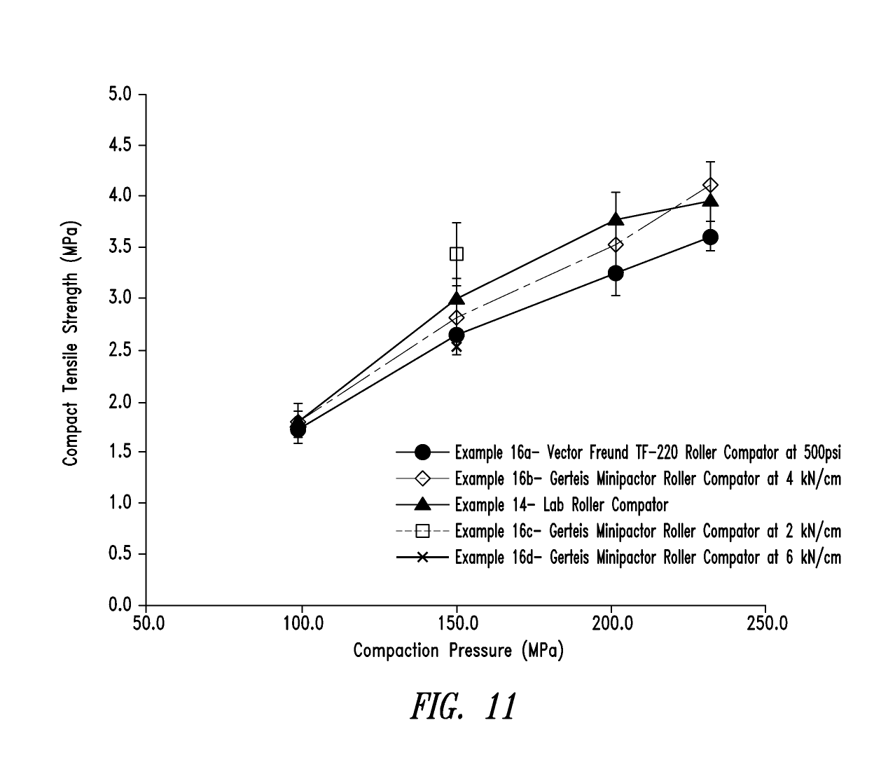

The compressibility of the milled granules prepared by the lab-scale roller compactor in Example 14 was compared to the compressibility of the granules prepared by large-scale processing equipment available from commercial vendors. The results are shown in FIG. 11. The compressibility of the granules from Example 14 was found to be comparable to the granules manufactured using Vector-Freund TF-220 at 500 psi roller pressure (Example 16a) and Gerteis Minipactor at 4 kN/cm roller pressure (Example 16b). These results demonstrate the utility of the lab-scale roller compactor in generating a compaction pressure that is comparable to large-scale processing equipment.

### Example 17

A portion of the granules from Example 14 (cenicriviroc mesylate, fumaric acid, microcrystalline cellulose, cross-linked sodium carboxymethyl cellulose, and magnesium stearate) were blended with extragranular lamivudine (3TC), microcrystalline cellulose, cross-linked sodium carboxymethyl cellulose, and magnesium stearate and compressed into tablets having a hardness greater than 6 kP and friability less than 0.8% w/w. The resulting powder blend and tablets had the composition shown in Table 18.

TABLE 18
(25/300 CVC/3TC)

| | Concentration | Mass (mg) per |
|---|---|---|
| Ingredient | (% w/w) | tablet |
| Cenicriviroc Mesylate | 5.69 | 28.45^{a} |
| Lamivudine | 60.00 | 300.00 |
| Fumaric Acid | 5.33 | 26.67 |
| Microcrystalline | 22.16 | 110.82 |
| Cellulose | | |
| Cross-linked Sodium | 5.65 | 28.25 |
| Carboxymethyl | | |
| Cellulose | | |
| Magnesium Stearate | 1.16 | 5.81 |
| Total | 100.0 | 500.0 |

^{a}Equivalent to 25 mg cenicriviroc freebase.

### Example 18

A portion of the granules from Example 14 (cenicriviroc mesylate, fumaric acid, microcrystalline cellulose, cross-linked sodium carboxymethyl cellulose, and magnesium stearate) was blended with extragranular lamivudine, microcrystalline cellulose, cross-linked sodium carboxymethyl cellulose, and magnesium stearate and compressed into tablets. The resulting powder blend and tablets had the composition shown in Table 19.

TABLE 19
(75/300 CVC/3TC)

| | Concentration | Mass (mg) per |
|---|---|---|
| Ingredient | (% w/w) | tablet |
| Cenicriviroc Mesylate | 13.13 | 85.35^{a} |
| Lamivudine | 46.15 | 300.00 |
| Fumaric Acid | 12.31 | 80.00 |
| Microcrystalline | 20.54 | 133.46 |
| Cellulose | | |
| Cross-linked Sodium | 6.50 | 42.25 |
| Carboxymethyl | | |
| Cellulose | | |
| Magnesium Stearate | 1.38 | 8.94 |
| Total | 100.0 | 650.0 |

^{a}Equivalent to 75 mg cenicriviroc freebase.

### Example 19

A portion of the granules from Example 14 (cenicriviroc mesylate, fumaric acid, microcrystalline cellulose, cross-linked sodium carboxymethyl cellulose, and magnesium stearate) was blended with extragranular lamivudine, microcrystalline cellulose, cross-linked sodium carboxymethyl cellulose, and magnesium stearate and compressed into tablets having a hardness greater than 10 kP and friability less than 0.8% w/w. The resulting tablets had the composition shown in Table 20.

TABLE 20
(150/300 CVC/3TC)

| | Concentration | Mass (mg) per |
|---|---|---|
| Ingredient | (% w/w) | tablet |
| Cenicriviroc Mesylate | 17.97 | 170.69^{a} |
| Lamivudine | 31.58 | 300.00 |
| Fumaric Acid | 16.84 | 160.00 |
| Microcrystalline | 24.78 | 235.43 |
| Cellulose | | |
| Cross-linked Sodium | 7.31 | 69.50 |
| Carboxymethyl | | |
| Cellulose | | |
| Magnesium Stearate | 1.51 | 14.38 |
| Total | 100.0 | 950.0 |

^{a}Equivalent to 150 mg cenicriviroc freebase.

### Example 20

A portion of the granules from Example 15 (cenicriviroc mesylate, fumaric acid, microcrystalline cellulose, cross-linked sodium carboxymethyl cellulose, and magnesium stearate) were blended with extragranular lamivudine, microcrystalline cellulose, cross-linked sodium carboxymethyl cellulose, and magnesium stearate and compressed into tablets having a hardness greater than 10 kP and friability less than 0.8% w/w. The resulting tablets had the composition shown in Table 21.

TABLE 21
(150/300 CVC Concentrated/3TC)

| | Concentration | Mass (mg) per |
|---|---|---|
| Ingredient | (% w/w) | tablet |
| Cenicriviroc Mesylate | 21.34 | 170.69^{a} |
| Lamivudine | 37.50 | 300.00 |
| Fumaric Acid | 20.00 | 160.00 |
| Microcrystalline | 12.01 | 96.01 |
| Cellulose | | |
| Cross-linked Sodium | 7.64 | 61.10 |
| Carboxymethyl | | |
| Cellulose | | |
| Magnesium Stearate | 1.53 | 12.20 |
| Total | 100.0 | 800.0 |

^{a}Equivalent to 150 mg cenicriviroc freebase.

### Example 21

### Composition Containing Intra-Granular (IG) Cenicriviroc and Half IG/Half Extra-Granular (EG) Lamivudine

In this example, granules were prepared as described in Example 14 except that the granules also contained a half of the desired amount of lamivudine. The granules were blended with the remaining portion of lamivudine, microcrystalline cellulose, cross-linked sodium carboxymethyl cellulose, and magnesium stearate and the powder blend compressed into tablets. That is, half the amount of lamivudine was present in the intra-granular portion and the remaining half of lamivudine was present in the extra-granular portion. The resulting powder blend and tablets had the composition shown in Table 22.

TABLE 22

| | Concentration | Mass (mg) per |
|---|---|---|
| Ingredient | (% w/w) | tablet |
| Cenicriviroc Mesylate | 22.76 | 170.69^{a} |
| Lamivudine | 40.00 | 300.00 |
| Fumaric Acid | 21.33 | 160.00 |
| Microcrystalline | 5.82 | 43.66 |
| Cellulose | | |
| Cross-linked Sodium | 8.55 | 64.15 |
| Carboxymethyl | | |
| Cellulose | | |
| Magnesium Stearate | 1.53 | 11.15 |
| Total | 100.0 | 750.0 |

^{a}Equivalent to 150 mg cenicriviroc freebase.

### Example 22

In this example, granules were prepared as described in Example 14 except that the granules contained the entire amount of lamivudine. That is, lamivudine was present solely in the IG portion. The granules were blended with microcrystalline cellulose, cross-linked sodium carboxymethyl cellulose, and magnesium stearate and compressed into tablets. The resulting powder blend and tablets had the composition shown in Table 23.

TABLE 23

| | Concentration | Mass (mg) per |
|---|---|---|
| Ingredient | (% w/w) | tablet |
| Cenicriviroc Mesylate | 22.76 | 170.69^{a} |
| Lamivudine | 40.00 | 300.00 |
| Fumaric Acid | 21.33 | 160.00 |
| Microcrystalline | 6.60 | 49.51 |
| Cellulose | | |
| Cross-linked Sodium | 7.61 | 57.10 |
| Carboxymethyl | | |
| Cellulose | | |
| Magnesium Stearate | 1.69 | 12.70 |
| Total | 100.0 | 750.0 |

^{a}Equivalent to 150 mg cenicriviroc freebase.

### Example 23

A portion of the granules from Example 14 (cenicriviroc mesylate, fumaric acid, microcrystalline cellulose, cross-linked sodium carboxymethyl cellulose, and magnesium stearate) was blended with extragranular microcrystalline cellulose, cross-linked sodium carboxymethyl cellulose, and magnesium stearate to obtain a powder blend comprising cenicriviroc granules. Lamivudine was blended separately with microcrystalline cellulose, cross-linked sodium carboxymethyl cellulose, and magnesium stearate to obtain a powder blend comprising lamivudine. A bilayer tablet was prepared using the powder blend comprising cenicriviroc granules and the powder blend comprising lamivudine. The resulting bilayer tablet had the composition shown in Table 24.

TABLE 24

| | Concentration | Mass (mg) per |
|---|---|---|
| Ingredient | (% w/w) | tablet |
| CVC Layer | | |
| Cenicriviroc Mesylate | 18.96 | 170.69^{a} |
| Fumaric Acid | 17.78 | 160.00 |
| Microcrystalline | 20.53 | 184.78 |
| Cellulose | | |
| Cross-linked Sodium | 4.34 | 39.00 |
| Carboxymethyl | | |
| Cellulose | | |
| Magnesium Stearate | 1.17 | 10.53 |
| 3TC Layer | | |
| Lamivudine | 33.33 | 300.00 |
| Microcrystalline | 2.85 | 25.62 |
| Cellulose | | |
| Cross-linked Sodium | 0.74 | 6.70 |
| Carboxymethyl | | |
| Cellulose | | |
| Magnesium Stearate | 0.30 | 2.68 |
| Total | 100.0 | 900.0 |

^{a}Equivalent to 150 mg cenicriviroc freebase.

### Example 24

The absolute bioavailability of the tablets of Examples 18-23 (containing a combination of cenicriviroc and 3TC) and Example 14 (containing cenicriviroc as a single active agent) was tested in fasted, untreated beagle dogs. All tablets were scaled down to deliver a constant dose of cenicriviroc of 25 mg with the corresponding proportional decrease in lamivudine to either 100 mg for Example 18 or 50 mg for Examples 19-23. The absolute bioavailability results are summarized in Table 25. The bioavailability of the tablets of Example 14 (cenicriviroc as a single agent) and Examples 19-20 (combination of cenicriviroc and 3TC) was also tested under pentagastrin pre-treatment state which induces the lowest gastric pH resembling the pH conditions of the human stomach.

TABLE 25

| | | |
|---|---|---|
| | CVC Absolute | 3TC Absolute |
| | Bioavail- | Bioavail- |
| | ability (%) | ability (%) |
| Component | n = 5 dogs | n = 5 dogs |

Fasted, No Pretreatment (gastric pH 2.0-4.0), n = 5

| | | |
|---|---|---|
| Example 14 CVC Tablet - Lab | 18.1 | N/A |
| Roller Compactor | | |
| Example 14 CVC Tablet - Vector | 20.6 | N/A |
| TF-220 Roller Compactor | | |
| Example 14 CVC Tablet - Gerteis | 16.6 | N/A |
| Minipactor Roller Compactor | | |
| Example 18 | 13.2 | 103 |
| Example 19 | 18.6 | 95.8 |
| Example 20 | 12.0 | 90.2 |
| Example 21 | 18.8 | 108 |
| Example 22 | 13.8 | 100 |
| Example 23 | 13.7 | 126 |

Fasted, Pentagastrin Pretreatment (gastric pH 1.0-2.5), n = 5

| | | |
|---|---|---|
| Example 14 CVC Tablet - Vector | 17.7 | N/A |
| TF-220 Roller Compactor^{a} | | |
| Example 19 | 18.5 | 107 |
| Example 20 | 22.1 | 96.3 |

^{a}50 mg dose of cenicriviroc.

The absolute bioavailability data shows that the exposure of CVC obtained using the combination formulations of Examples 19 and 21 is comparable to the CVC single agent formulation of Example 14. The bioavailability data for Example 19 with and without the pentagastrin pretreatment showed that the level of CVC exposure is comparable regardless of gastric pH conditions. More importantly, the data also shows that the acidic microenvironment functionality of the CVC formulation is maintained in this combination product formulation. The data for Example 21 (½ IG ½ EG 3TC) shows that even when half the amount of 3TC, which is weakly basic, was in direct contact with CVC/fumaric acid granules (IG), the exposure of CVC and 3TC obtained was comparable to that of Example 19 where 3TC was completely located extragranularly (EG) with less intimate contact with CVC/fumaric acid. This data indicates that 3TC, which is highly water soluble, dissolved at a rate faster than that of fumaric acid, which is used in the invention as a slow to dissolve solubilizer for CVC thereby eliminating the possibility that weakly basic 3TC would neutralize fumaric acid. The data also confirms that the acidic microenvironment feature of the invention based on the slow to dissolve fumaric acid excipient provides desired CVC release characteristics in vivo despite the presence of 3TC, a weakly basic drug. Example 20, where granules prepared using a concentrated CVC formulation were used, shows only 12.0% CVC exposure under no pretreatment and 22.1% with lower gastric pH conditions. The exposure values for Examples 18, 20, 22 and 23 are still acceptable and may or may not require a dose adjustment if administered to human subjects to compare relative bioavailability. The absolute bioavailability for lamivudine which is greater than 90% for all formulations is acceptable and appears to be independent of formulation composition and manufacturing process.

### Example 25

The disintegration behavior of CVC/3TC tablets was characterized by placing a single tablet of each sample prepared for dog pharmacokinetic evaluation in approximately 250 mL water and observing the mode and speed of disintegration.

Table 26 summarizes the disintegration results for Examples 18 and 20-22. Tablets of Examples 18 and 20 which contained the entire quantity of lamivudine extragranularly displayed rapid disintegration similar to lamivudine active ingredient compressed as a tablet. Examples 21-22 where half or the entire amount of lamivudine was present intragranularly displayed unexpected disintegration pattern. Specifically, Example 21 with half the amount of lamivudine present intragranularly disintegrated slowly over a period of several minutes. Example 22 with the entire quantity of lamivudine present intragranularly did not disintegrate at all. These results were unexpected given the high aqueous solubility of lamivudine at 70 mg/mL. It is possible that the interaction between the intragranular components may prohibit proper wetting and disintegration of the tablet and granules. Even though the addition of lamivudine in the intragranular portion of the cenicriviroc granulation is a strategy to conserve the tablet mass, special consideration with respect to biopharmaceutical performance must be given due to the tablet disintegration behavior changes.

TABLE 26

| | Tablet | | | |
|---|---|---|---|---|
| | Weight | | Compression | Disintegration |
| Sample | (mg) | Tooling | Force (lb) | Observations |
| Lamivudine | 150 mg | ¼-inch round, | 800 | Rapid |
| Active | | standard concave | | immediate |
| Ingredient | | | | disintegra- |
| | | | | tion <30 |
| | | | | seconds |
| Example 18 | 218 mg | ⅜-inch round, | 800 | Rapid |
| | | standard concave | | immediate |
| | | | | disintegra- |
| | | | | tion <30 |
| | | | | seconds |
| Example 20 | 133 mg | ¼-inch round, | 1000 | Rapid |
| | | standard concave | | immediate |
| | | | | disintegra- |
| | | | | tion <30 |
| | | | | seconds |
| Example 21 | 125 mg | ¼-inch round, | 1000 | Slow, erosion |
| | | standard concave | | disintegra- |
| | | | | tion |
| | | | | Approx 2-3 |
| | | | | minutes |
| Example 22 | 127 mg | ¼-inch round, | 1000 | No disintegra- |
| | | standard concave | | tion >30 |
| | | | | minutes |

### Example 26

The CVC/3TC tablets of Examples 17, 19, and 20 and the CVC single agent tablets of Example 14 were tested for total impurities under accelerated stability conditions by exposing the tablets to an environment of 75% relative humidity at 40° C. All tablets were packaged in HDPE bottles, with induction seal, and a desiccant during the study. As summarized in Tables 27a and 27b, the CVC/3TC tablets of Examples 17, 19, and 20 were as stable as CVC single agent tablets of Example 14 and commercial 3TC single agent tablets Epivir with not more than 0.1% increase in impurities or degradants over 9 weeks of accelerated storage. This indicates that the active ingredients were sufficiently chemically compatible and stable in the formulations and processes described above. No lamivudine impurities or degradants were observed in any of the examples as shown in Table 27b.

TABLE 27a

| | Example 14 | Example 17 | Example 19 | Example 20 |
|---|---|---|---|---|
| | Total CVC | Total CVC | Total CVC | Total CVC |
| Time | Impurities | Impurities | Impurities | Impurities |
| (Weeks) | (%) | (%) | (%) | (%) |
| 0 | 1.2 | 1.4 | 1.6 | 1.3 |
| 2 | 1.1 | 1.5 | 1.4 | 1.4 |
| 6 | 1.0 | N/D | N/D | N/D |
| 9 | N/D | 1.5 | 1.4 | 1.4 |
| 12 | 1.0 | N/D | N/D | N/D |

N/D—not determined

TABLE 27b

| | Example 17 | Example 19 | Example 20 |
|---|---|---|---|
| | Total 3TC | Total 3TC | Total 3TC |
| Time | Impurities | Impurities | Impurities |
| (Weeks) | (%) | (%) | (%) |
| 0 | BLQ | BLQ | BLQ |
| 2 | BLQ | BLQ | BLQ |
| 9 | BLQ | BLQ | BLQ |

BLQ—below the limit of quantitation (<0.05%)

### Example 27

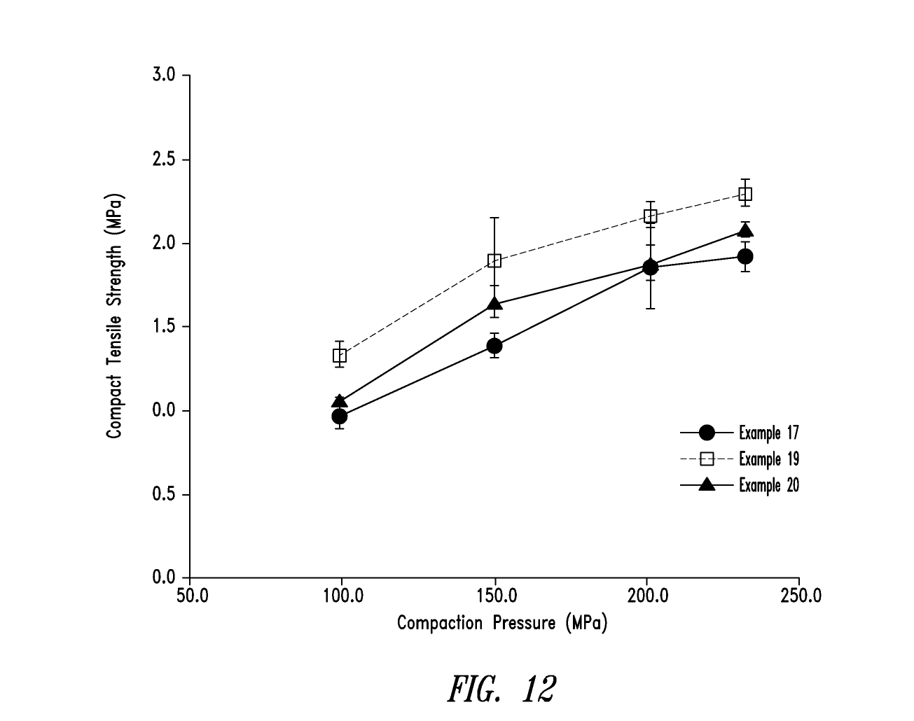

Compression profiles of CVC/3TC powder blends of Examples 17, 19, and 20 were measured and are shown in FIG. 12. Although the addition of 3TC decreased the compressibility of the CVC single agent powder blends shown in FIG. 10, all CVC/3TC powder blends still showed acceptable compressibility characteristics required for commercial product purposes. Lamivudine is a highly crystalline brittle material with large discrete particles that disrupt the powder matrix undergoing the compaction process. Examples 17 and 20 with higher concentrations of lamivudine exhibit lower compressibility than Example 19 containing 150 mg of more excipient mass than Example 20.

### Example 28

Bilayer tablets comprising a combination of three active agents, CVC, 3TC, and efavirenz (EFV), were prepared for Single Tablet Regimen (STR) treatment studies of HIV. In the bilayer tablets, the CVC/3TC combination exists as a single layer whereas the third active agent, EFV, exists as a second layer. The CVC/3TC layer of the tablet was prepared using the concentrated composition of Example 20. However, any of the CVC/3TC combinations disclosed above or related variations could be similarly used in this STR tablet configuration.

The EFV layer was prepared by a conventional high-shear wet granulation process using a 5 L stainless steel granulator bowl. EFV, microcrystalline cellulose, cross-linked sodium carboxymethyl cellulose, sodium lauryl sulfate, and hydroxypropyl cellulose were blended in a high-shear mixer for 2 minutes at speed setting #2 to prepare a 300 g batch. To the blend, 238 ml of purified water was added over approximately 6 minutes to obtain suitable granulation and further blended, if necessary. The granules were milled with a blade forward hammer mill and dried in a tray dryer at 80° C. The dried granules were further milled and blended with magnesium stearate. The EFV layer weight of the bilayer tablet was 850 mg corresponding to 600 mg of EFV active ingredient and 250 mg of excipients. Separate layers of CVC/3TC and EFV were compressed into bilayer tablets having a hardness greater than 15 kP and a friability of less than 0.8% w/w. The bilayer tablets had the composition shown in Table 28.

TABLE 28
(CVC/EFV/3TC Single Tablet Regimen-1)

| | Concentration | Mass (mg) per |
|---|---|---|
| Ingredient | (% w/w) | tablet |
| CVC/3TC Layer | | |
| Cenicriviroc Mesylate | 10.34 | 170.69^{a} |
| Lamivudine | 18.18 | 300.00 |
| Fumaric Acid | 9.70 | 160.00 |
| Microcrystalline | 5.82 | 96.01 |
| Cellulose | | |
| Cross-linked Sodium | 3.70 | 61.10 |
| Carboxymethyl | | |
| Cellulose | | |
| Magnesium Stearate | 0.74 | 12.2 |
| EFV Layer | | |
| Efavirenz | 36.36 | 600.00 |
| Microcrystalline | 7.97 | 131.50 |
| Cellulose | | |
| Cross-linked Sodium | 3.64 | 60.00 |
| Carboxymethyl | | |
| Cellulose | | |
| Sodium Lauryl Sulfate | 0.73 | 12.00 |
| Hydroxypropyl | 2.30 | 38.00 |
| Cellulose | | |
| Magnesium Stearate | 0.52 | 8.50 |
| Total | 100.0 | 1650.0 |

^{a}Equivalent to 150 mg cenicriviroc freebase.

### Example 29

A bilayer tablet comprising CVC, 3TC, and EFV as active agents was prepared as described in Example 28 except that the weight of the EFV layer was 775 mg. The CVC/3TC layer of the tablet was prepared using the concentrated composition of Example 20. However, any of the CVC/3TC combinations disclosed above or related variations could be similarly used in this STR tablet configuration. Tablets had a hardness greater than 15 kP and a friability of less than 0.8% w/w. The bilayer tablets had the composition shown in Table 29.

TABLE 29
(CVC/EFV/3TC Single Tablet Regimen-2)

| | Concentration | Mass (mg) per |
|---|---|---|
| Ingredient | (% w/w) | tablet |
| CVC/3TC Layer | | |
| Cenicriviroc Mesylate | 10.84 | 170.69^{a} |
| Lamivudine | 19.05 | 300.00 |
| Fumaric Acid | 10.16 | 160.00 |
| Microcrystalline | 6.10 | 96.01 |
| Cellulose | | |
| Cross-linked Sodium | 3.88 | 61.10 |
| Carboxymethyl | | |
| Cellulose | | |
| Magnesium Stearate | 0.77 | 12.2 |
| EFV Layer | | |
| Efavirenz | 38.09 | 600.00 |
| Microcrystalline | 3.82 | 60.20 |
| Cellulose | | |
| Cross-linked Sodium | 3.81 | 60.00 |
| Carboxymethyl | | |
| Cellulose | | |
| Sodium Lauryl Sulfate | 0.76 | 12.00 |
| Hydroxypropyl | 2.22 | 35.00 |
| Cellulose | | |
| Magnesium Stearate | 0.50 | 7.80 |
| Total | 100.0 | 1575.0 |

^{a}Equivalent to 150 mg cenicriviroc freebase.

### Example 30

The absolute bioavailability of the CVC/3TC/EFV tablets of Examples 28-29 was measured in fasted, pentagastrin-pretreated beagle dogs and was compared to that of the CVC single agent tablets of Example 14. All tablets were scaled down to deliver a constant dose of 25 mg cenicriviroc free base with the corresponding proportional decrease in lamivudine to deliver a dose of 50 mg, and in efavirenz to deliver a dose of 100 mg. The absolute bioavailability results are summarized in Table 30.

TABLE 30

| | CVC Absolute | 3TC Absolute | |
|---|---|---|---|
| | Bioavail- | Bioavail- | |
| Component | ability (%) | ability (%) | EFV Absolute |
| Fasted, Pentagastrin Pretreatment | | | Bioavail- |
| (gastric pH 1.0-2.5), n = 5 | | | ability (%) |
| Example 14 CVC | 17.7 | N/A | N/A |
| Tablet - Vector | | | |
| TF-220 Roller | | | |
| Compactor^{a} | | | |
| Example 28 | 5.9 | 81.3 | 16.5 |
| Example 29 | 3.9 | 107 | 16.5 |

^{a}50 mg dose of cenicriviroc

The absolute bioavailability data shows a considerable reduction in the exposure of CVC when administered in the presence of efavirenz. Efavirenz is a known inducer of hepatic enzyme CYP3A4 and it has been shown that efavirenz increases the metabolism of cenicriviroc in humans thereby decreasing the cenicriviroc plasma concentration by approximately 2-fold.

### Example 31

The tablets of Examples 28 and 29 were tested for total impurities under accelerated stability conditions by exposing the tablets to an environment of 75% relative humidity at 40° C. All tablets were packaged with a desiccant in induction sealed HDPE bottles. As summarized in Table 31, CVC total impurities showed no significant change over 4 weeks of accelerated storage conditions. No lamivudine impurities were measured in either of the examples as shown in Table 31. Additionally, Table 17 shows no significant change in efavirenz degradation products.

TABLE 31

| | Example 28 | Example 29 | Example 28 | Example 29 | Example 28 | Example 29 |
|---|---|---|---|---|---|---|
| | Total CVC | Total CVC | Total 3TC | Total 3TC | Total EFV | Total EFV |
| Time | Impurities | Impurities | Impurities | Impurities | Impurities | Impurities |
| (Weeks) | (%) | (%) | (%) | (%) | (%) | (%) |
| 0 | 1.3 | 1.2 | BLQ | BLQ | 0.1 | 0.1 |
| 4 | 1.3 | 1.3 | BLQ | BLQ | 0.2 | 0.2 |

BLQ—below the limit of quantitation (<0.05%).

### Example 32

Tablets of Examples 28-29 and tablets of Examples 17, 19, and 20 were tested for strength and water content under accelerated stability conditions by exposing the packaged tablets to an environment of 75% relative humidity at 40° C. As summarized in Tables 32-33 below, no significant change was observed in the strength of CVC and 3TC in the tablets of Examples 19 and 20 and the STR tablets of Examples 28-29. Tablets of Example 17 did not show any significant change after 2 weeks, but showed a numerical decrease in the strength of CVC and 3TC after 4 weeks. Additional testing confirmed that this decrease was not significant and arose as a result of an artifact in the analytical testing method.

TABLE 32
Strength under accelerated conditions (40° C./75% RH)

| | Example 17 | | Example 19 | | Example 20 | |
|---|---|---|---|---|---|---|
| Time | n = 5 | | n = 5 | | n = 5 | |
| (weeks) | 3TC (% LC) | CVC | 3TC (% LC) | CVC | 3TC (% LC) | CVC (% LC) |
| 0 | 100.0 ± 0.9 | 100.3 ± 4.2 | 99.9 ± 1.9 | 99.1 ± 2.2 | 96.4 ± 1.8 | 102.7 ± 1.9 |
| 2 | 98.8 ± 1.4 | 97.6 ± 5.2 | 98.1 ± 1.6 | 98.6 ± 2.0 | 98.1 ± 1.1 | 98.0 ± 1.8 |
| 4 | 95.8 ± 9.0 | 92.3 ± 5.5 | 100.4 ± 2.6 | 99.9 ± 3.0 | 100.5 ± 2.7 | 99.8 ± 4.0 |

TABLE 33
Strength under accelerated conditions (40° C./75% RH)

| Time | Example 28 | | | Example 29 | | |
|---|---|---|---|---|---|---|
| (weeks) | 3TC (% LC) | EFV (% LC) | CVC | 3TC (% LC) | EFV (% LC) | CVC |
| 0 | 98.5 ± 1.4 | 102.2 ± 0.7 | 97.4± | 97.0 ± 1.9 | 101.7 ± 0.9 | 98.8 ± 1.7 |
| 4 | 98.9 ± 1.2 | 101.2 ± 0.4 | 101.9± | 98.5 ± 1.1 | 101.2 ± 0.5 | 102.2 ± 1.3 |

Table 34 shows that no significant change in the water content as determined by Karl Fischer was observed for any of the CVC/3TC tablets of Examples 17, 19, and 20 and STR tablets of Examples 28-29 after 4 weeks of storage at 40° C./75% RH.

TABLE 34
Water content under accelerated conditions (40° C./75% RH)

| | T = 0 | | T = 2 Weeks | | T = 4 Weeks | |
|---|---|---|---|---|---|---|
| Sample | % water | Average | % water | Average | % water | Average |
| Example 17 | 0.4631 | 0.48 | 0.3745 | 0.43 | 0.3949 | 0.44 |
| | 0.4884 | | 0.4796 | | 0.4815 | |
| Example 19 | 0.8434 | 0.85 | 0.7978 | 0.84 | 0.8538 | 0.85 |
| | 0.8629 | | 0.8889 | | 0.8502 | |
| Example 20 | 0.4173 | 0.42 | 0.3975 | 0.42 | 0.3920 | 0.39 |
| | 0.4288 | | 0.4350 | | 0.3945 | |
| Example 28 | 0.4650 | 0.49 | | | 0.4323 | 0.47 |
| | 0.5124 | | | | 0.5017 | |
| Example 29 | 0.3423 | 0.37 | | | 0.3763 | 0.38 |
| | 0.3991 | | | | 0.3817 | |
| | 1.1898 | | 1.1280 | | 1.2239 | |

Tablets of Examples 17, 19, and 20 were tested for dissolution after 9 weeks of storage at 40° C./75% RH. No significant change was observed in the dissolution profile for 3TC and CVC during 9 weeks of storage at 40° C./75% RH.

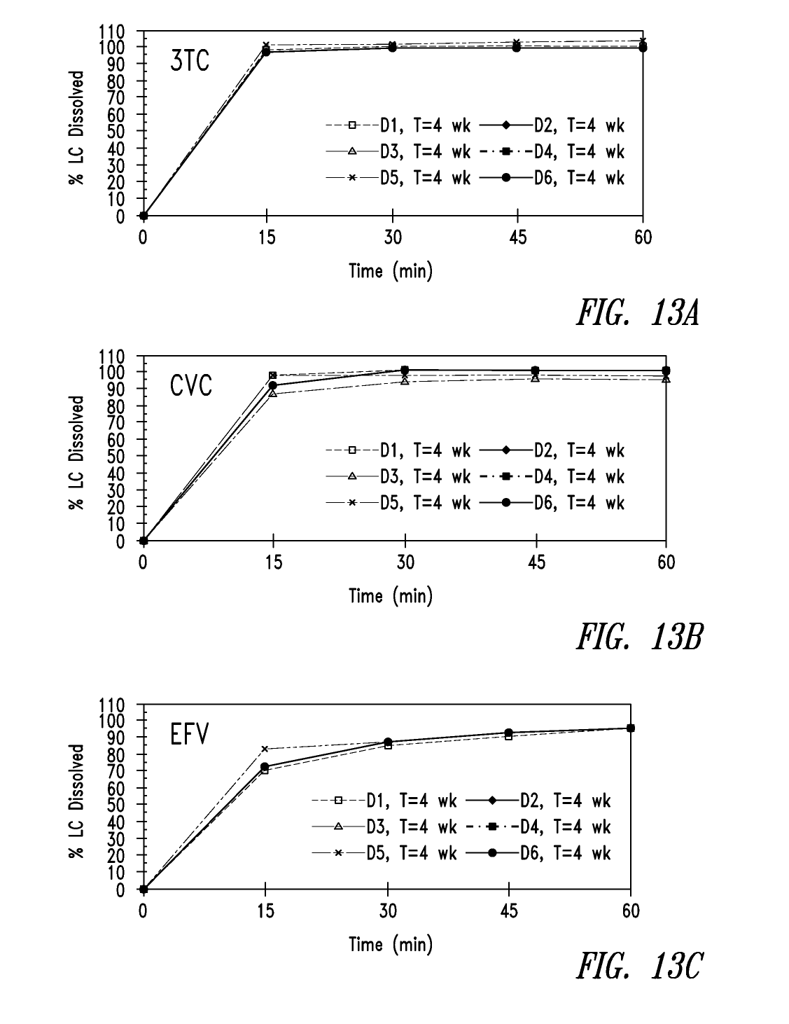

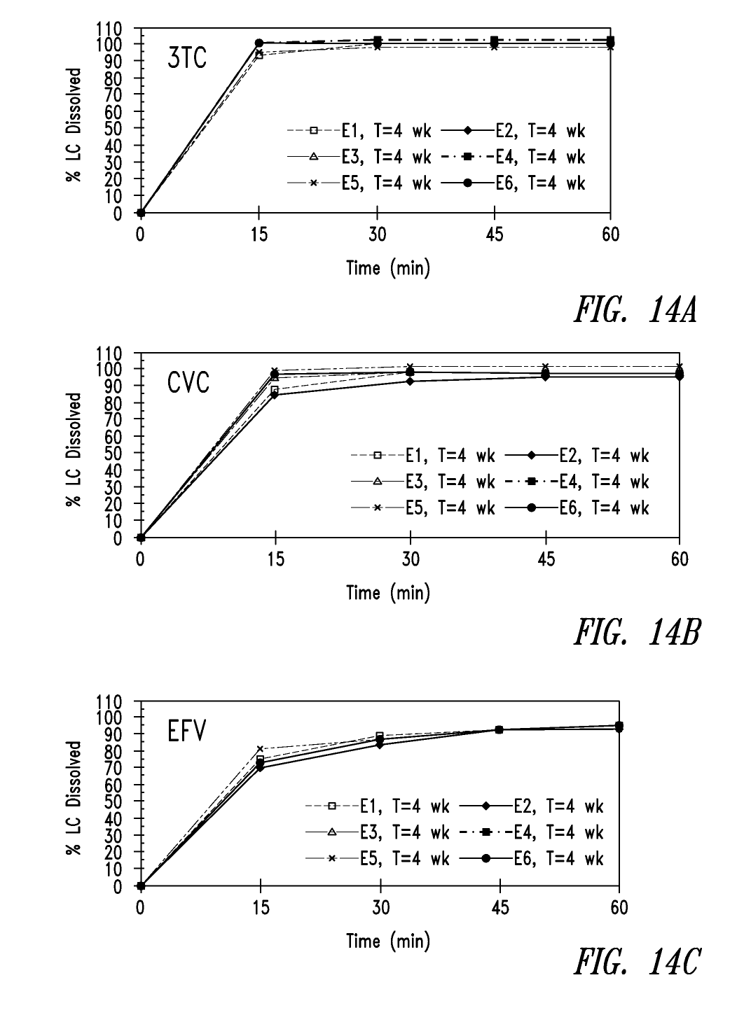

Tablets of Examples 28-29 were also tested for dissolution after 4 weeks of storage at 40° C./75% RH. The dissolution data is summarized in FIGS. 13-14.

### Example 33

Tablets of Examples 17, 19, and 20 were also tested for the formation of related substances after 9 weeks of storage at 40° C./75% RH. For this testing, a single tablet of Example 17 was placed in a 100 ml flask, 5 ml MiliQ water was added, the flask was placed on a shaker for 30 minutes at 200 rpm followed by the addition of 65 ml of methanol. The flask was placed back on a shaker for additional 30 minutes at 200 rpm and the contents were diluted to 100 ml with methanol. For tablets of Examples 19 and 20, a HPLC sample was prepared by placing a single tablet in a 500 ml flask, 25 ml MiliQ water was added, the flask was placed on a shaker for 30 minutes at 200 rpm, 325 ml of methanol was added, the flask was placed on shaker for additional 30 minutes at 200 rpm and the contents were diluted to 500 ml using methanol. The samples were analyzed for the formation of related substances using HPLC. CVC related substances increased from <LOQ (0.05%) to approximately 0.2% after 9 weeks of storage at 40° C./75% RH. No 3TC related substances were observed at levels greater than LOQ (0.05%) after 9 weeks of storage at 40° C./75% RH.

The related substance HPLC method parameters are listed in the table below:

TABLE 35

| Instrument | | | |
|---|---|---|---|
| Parameters | Description | | |
| Column | Waters XSelect HSS PFP, 3.5 μm, 4.6 × 150 mm | | |
| Detection | CVC: 293 nm | | |
| | 3TC: 270 nm | | |
| Column | 30° C. | | |
| Temperature | | | |
| Flow Rate | 0.8 mL/min | | |
| Injection | 3TC: 10 μL | | |
| Volume | CVC: 20 μL | | |
| Needle Wash | Open LC vial containing ≧1.5 mL of 90/10 ACN/Water | | |
| Run Time | 105 minutes | | |
| Mobile Phase | A 10 mM Ammonium Acetate in MilliQ Water, pH 5.5 | | |
| (MP) | B 95/5 Methanol/Acetonitrile | | |
| Mobile Phase | Time | | |
| Gradient | (minutes) | % A | % B |
| | 0 | 97 | 3 |
| | 5 | 97 | 65 |
| | 50 | 30 | 70 |
| | 65 | 30 | 70 |
| | 80 | 20 | 80 |
| | 100 | 20 | 80 |
| | 101 | 97 | 3 |
| | 105 | 97 | 3 |

### Example 34

The pharmacokinetic profile of the tablets of Example 28 (containing a combination of cenicriviroc, 3TC, and EFV) was tested in fasted, pentagastrin-treated beagle dogs. All tablets were scaled down to deliver a constant dose of 25 mg CVC, 50 mg 3TC, and 100 mg EFV. The results are summarized in Table 36.

TABLE 36

| | Dose | Dose | Cmax | Tmax | AUCₗₐₛₜ | AUC_{INF} | % | T_{1/2} | MRTₗₐₛₜ |
|---|---|---|---|---|---|---|---|---|---|
| Dog ID | (mg/dog) | (mg/kg) | (ng/mL) | (hr) | (ng/mL*hr) | (ng/mL*hr) | AUCextra | (hr) | (hr) |
| D101 | 25.0 | 2.68 | 83.8 | 2.00 | 563 | 599 | 6.07 | 5.91 | 7.17 |
| D103 | 25.0 | 2.71 | 74.5 | 2.00 | 371 | 384 | 3.24 | 5.04 | 5.98 |
| D104 | 25.0 | 2.27 | 4.88 | 2.00 | 19.5 | 23.3 | 16.1 | 2.35 | 3.85 |
| D106 | 25.0 | 2.59 | 31.3 | 2.00 | 183 | 196 | 6.49 | 6.44 | 6.48 |
| D108 | 25.0 | 2.58 | 32.9 | 2.00 | 193 | 197 | 1.97 | 4.28 | 5.76 |
| Mean | 25.0 | 2.57 | 45.5 | 2.00 | 266 | 280 | 6.77 | 4.80 | 5.85 |
| SD | 0.00 | 0.172 | 32.9 | 0.00 | 207 | 219 | 5.55 | 1.60 | 1.24 |
| CV % | 0.00 | 6.70 | 72.3 | 0.00 | 78.0 | 78.4 | 81.9 | 33.3 | 21.2 |

It should be understood that while the above description provides a person of ordinary skill in the art sufficient guidance to make, use, and practice the disclosure, it is not intended to be limiting. Various modifications can be made to this description without departing from the scope or spirit of the disclosure. Persons of ordinary skill may employ such variations as appropriate, and the disclosure may be practiced in ways other than those specifically described herein. For example, while some embodiments have been described with reference to specific types of inactive ingredients, such as fillers, disintegrants, and the like, one of ordinary skill in the art will recognize that other inactive ingredients can also be used to achieve similar results. Accordingly, the disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A composition comprising cenicriviroc or a salt thereof and fumaric acid.

2. The composition of claim 1, wherein the cenicriviroc or salt thereof is cenicriviroc mesylate.

3. The composition of claim 1 or 2, wherein the weight ratio of cenicriviroc or salt thereof to fumaric acid is from about 7:10 to about 10:7 based on the weight of free cenicriviroc.

4. The composition of any of claims 1-3, wherein the weight ratio of cenicriviroc or salt thereof to fumaric acid is from about 8:10 to about 10:8 based on the weight of free cenicriviroc.

5. The composition of any of claims 1-4, wherein the weight ratio of cenicriviroc or salt thereof to fumaric acid is from about 9:10 to about 10:9 based on the weight of free cenicriviroc.

6. The composition of any of claims 1-5, wherein the weight ratio of cenicriviroc or salt thereof to fumaric acid is from about 95:100 to about 100:95 based on the weight of free cenicriviroc.

7. The composition of any of claims 1-6, wherein the fumaric acid is present in an amount of from about 15% to about 40% by weight of the composition.

8. The composition of any of claims 1-7, wherein the fumaric acid is present in an amount of from about 20% to about 30% by weight of the composition.

9. The composition of any of claims 1-8, wherein the fumaric acid is present in an amount of about 25% by weight of the composition.

10. The composition of any of claims 1-9, wherein the cenicriviroc or salt thereof is present in an amount of from about 15% to about 40% by weight of the composition based on the weight of free cenicriviroc.

11. The composition of any of claims 1-10, wherein the cenicriviroc or salt thereof is present in an amount of from about 20% to about 30% by weight of the composition based on the weight of free cenicriviroc.

12. The composition of any of claims 1-11, wherein the cenicriviroc or salt thereof is present in an amount of about 25% by weight of the composition based on the weight of free cenicriviroc.

13. The composition of any of claims 1-12, further comprising one or more fillers.

14. The composition of claim 13, wherein the one or more fillers are selected from microcrystalline cellulose, calcium phosphate dibasic, cellulose, lactose, sucrose, mannitol, sorbitol, starch, and calcium carbonate.

15. The composition of any of claims 13-14, wherein the one or more fillers is microcrystalline cellulose.

16. The composition of any of claims 13-15, wherein the weight ratio of the one or more fillers to the cenicriviroc or salt thereof is from about 25:10 to about 10:8 based on the weight of free cenicriviroc.

17. The composition of any of claims 13-16, wherein the weight ratio of the one or more fillers to the cenicriviroc or salt thereof is from about 20:10 to about 10:10 based on the weight of free cenicriviroc.

18. The composition of any of claims 13-17, wherein the weight ratio of the one or more fillers to the cenicriviroc or salt thereof is about 15:10 based on the weight of free cenicriviroc.

19. The composition of any of claims 13-18, wherein the one or more fillers are present in an amount of from about 25% to about 55% by weight of the composition.

20. The composition of any of claims 13-19, wherein the one or more fillers are present in an amount of from about 30% by weight to about 50% by weight of the composition.

21. The composition of any of claims 13-20, wherein the one or more fillers are present in an amount of about 40% by weight of the composition.

22. The composition of any of claims 1-21, further comprising one or more disintegrants.

23. The composition of claim 22, wherein the one or more disintegrants are selected from cross-linked polyvinylpyrrolidone, cross-linked sodium carboxymethyl cellulose, and sodium starch glycolate.

24. The composition of any of claims 22-23, wherein the one or more disintegrants is cross-linked sodium carboxymethyl cellulose.

25. The composition of any of claims 22-24, wherein the weight ratio of the one or more disintegrants to the cenicriviroc or salt thereof is from about 10:100 to about 30:100 based on the weight of free cenicriviroc.

26. The composition of any of claims 22-25, wherein the weight ratio of the one or more disintegrants to the cenicriviroc or salt thereof is about 25:100 based on the weight of free cenicriviroc.

27. The composition of any of claims 22-26, wherein the one or more disintegrants are present in an amount of from about 2% to about 10% by weight of the composition.

28. The composition of any of claims 22-27, wherein the one or more disintegrants are present in an amount of from about 4% to about 8% by weight of the composition.

29. The composition of any of claims 22-28, wherein the one or more disintegrants are present in an amount of about 6% by weight of the composition.

30. The composition of any of claims 1-29, further comprising one or more lubricants.

31. The composition of claim 30, wherein the one or more lubricants are selected from stearin, magnesium stearate, and stearic acid.

32. The composition of any of claims 30-31, wherein the one or more lubricants is magnesium stearate.

33. The composition of any of claims 30-32, wherein the one or more lubricants are present in an amount of from about 0.25% to about 5% by weight of the composition.

34. The composition of any of claims 30-33, wherein the one or more lubricants are present in an amount of from about 0.75% to about 3% by weight of the composition.

35. The composition of any of claims 30-34, wherein the one or more lubricants are present in an amount of about 1.25% by weight of the composition.

36. The composition of any of claims 1-35, wherein the composition is substantially similar to that of Table 3a.

37. The composition of any of claims 1-35, wherein the composition is substantially similar to that of Table 3b.

38. The composition of any of claims 1-37, wherein the composition is produced by a process involving dry granulation.

39. The composition of any of claims 1-38, wherein the composition has a water content of no more than about 4% by weight after about six weeks of exposure to about 40° C. at about 75% relative humidity when packaged with a desiccant in a container.

40. The composition of any of claims 1-39, wherein the composition has a water content of no more than about 2% by weight after about six weeks of exposure to about 40° C. at about 75% relative humidity when packaged with a desiccant in a container.

41. The composition of any of claims 1-40, wherein the composition has a total impurity and degradant level of no more than about 2.5% after 12 weeks of exposure to about 40° C. at about 75% when packaged with a desiccant in a container.

42. The composition of any of claims 1-41, wherein the composition has a total impurity and degradant level of no more than about 1.5% after 12 weeks of exposure to about 40° C. at about 75% when packaged with a desiccant in a container.

43. The composition of any of claims 1-42, wherein the cenicriviroc or salt thereof has a mean absolute bioavailability after oral administration that is substantially similar to the mean absolute bioavailability of the cenicriviroc or salt thereof in a solution after oral administration.

44. The composition of any of claims 1-42, which exhibits an AUC of cenicriviroc that is about 200% or higher of the AUC of cenicriviroc exhibited by a reference solid formulation following oral administration.

45. The composition of any of claims 1-42, which exhibits a Cmax of cenicriviroc that is at least 50% higher than the Cmax of cenicriviroc exhibited by a reference solid formulation following oral administration.

46. The composition of any of claims 1-42, further comprising one or more additional pharmaceutically active agents.

47. The composition of claim 46, wherein the one or more additional pharmaceutically active agents is one or more additional antiretroviral drugs selected from CCR5 receptor antagonists, entry inhibitors, nucleoside reverse transcriptase inhibitors, nucleotide reverse transcriptase inhibitors, non-nucleoside reverse transcriptase inhibitors, protease inhibitors, integrase inhibitors, and maturation inhibitors.

48. The composition of any of claims 46-47, wherein the one or more additional pharmaceutically active agents are selected from maraviroc, lamivudine, efavirenz, raltegravir, vivecon, bevirimat, alpha interferon, zidovudine, abacavir, lopinavir, ritonavir, tenofovir, tenofovir disoproxil, tenofovir prodrugs, emtricitabine, elvitegravir, cobicistat darunavir, atazanavir, rilpivirine, and dolutegravir.

49. The composition of claim 48 comprising: cenicriviroc or a salt thereof and fumaric acid; and lamivudine.

50. The composition of claim 49, wherein the cenicriviroc or salt thereof is cenicriviroc mesylate.

51. The composition of claim 49 or 50, wherein the weight ratio of cenicriviroc or salt thereof to lamivudine is from about 1:15 to about 1:1 based on the weight of free cenicriviroc.

52. The composition of any of claims 49-51, wherein the weight ratio of cenicriviroc or salt thereof to lamivudine is from about 1:12 to about 2:3 based on the weight of free cenicriviroc.

53. The composition of any of claims 49-52, wherein the weight ratio of cenicriviroc or salt thereof to lamivudine is about 1:12; about 1:4; or about 1:2 based on the weight of free cenicriviroc.

54. The composition of any of claims 49-53, wherein lamivudine is present in an amount of from about 25% to about 65% by weight of the composition.

55. The composition of any of claims 49-54, wherein lamivudine is present in an amount of from about 30% to about 60% by weight of the composition.

56. The composition of any of claims 49-55, wherein lamivudine is present in an amount of about 31.6%; about 33.3%; about 37.5%; about 40.0%; about 46.2%; or about 60% by weight of the composition.

57. The composition of any of claims 49-56, comprising
about 15.8% cenicriviroc or salt thereof and about 31.6% lamivudine;
about 16.7% cenicriviroc or salt thereof and about 33.3% lamivudine;
about 18.8% cenicriviroc or salt thereof and about 37.5% lamivudine;
about 20% cenicriviroc or salt thereof and about 40.0% lamivudine;
about 11.5% cenicriviroc or salt thereof and about 46.2% lamivudine; or
about 5% cenicriviroc or salt thereof and about 60% lamivudine by weight of the composition and based on the weight of free cenicriviroc.

58. The composition of any of claims 49-57, further comprising one or more fillers.

59. The composition of claim 58, wherein the one or more fillers are selected from microcrystalline cellulose, calcium phosphate dibasic, cellulose, lactose, sucrose, mannitol, sorbitol, starch, and calcium carbonate.

60. The composition of claim 58 or 59, wherein the one or more fillers is microcrystalline cellulose.

61. The composition of any of claims 58-60, wherein the weight ratio of the one or more fillers to the cenicriviroc or salt thereof is from about 5:1 to about 1:5 based on the weight of free cenicriviroc.

62. The composition of any of claims 58-61, wherein the weight ratio of the one or more fillers to the cenicriviroc or salt thereof is about 1:4 to about 1:5; or about 2:3 to about 1:2: or about 2:1 to about 4:3; or about 5:1 to about 5:2 based on the weight of free cenicriviroc.

63. The composition of any of claims 58-62, wherein the one or more fillers are present in an amount of from about 5% to about 30% by weight of the composition.

64. The composition of any of claims 58-63, wherein the one or more fillers are present in an amount of about 5.8%%; about 6.6%; about 12%; about 20.5%; about 22.2%; about 23.4%; or about 24.8% by weight of the composition.

65. The composition of any of claims 58-64, comprising
about 15.8% cenicriviroc or salt thereof, about 31.6% lamivudine, and 24.8% one or more fillers;
about 16.7% cenicriviroc or salt thereof, about 33.3% lamivudine, and 23.4% one or more fillers;
about 18.8% cenicriviroc or salt thereof, about 37.5% lamivudine, and 12.0% one or more fillers;
about 20% cenicriviroc or salt thereof, about 40.0% lamivudine, and 5.8% one or more fillers;
about 20% cenicriviroc or salt thereof, about 40.0% lamivudine, and 6.6% one or more fillers;
about 11.5% cenicriviroc or salt thereof, about 46.2% lamivudine, and 20.5% one or more fillers; or
about 5% cenicriviroc or salt thereof, about 60% lamivudine, and 22.2% one or more fillers by weight of the composition and based on the weight of free cenicriviroc.

66. The composition of any of claims 49-65, further comprising one or more disintegrants.

67. The composition of claim 66, wherein the one or more disintegrants are selected from cross-linked polyvinylpyrrolidone, cross-linked sodium carboxymethyl cellulose, and sodium starch glycolate.

68. The composition of claim 66 or 67, wherein the one or more disintegrants is cross-linked sodium carboxymethyl cellulose.

69. The composition of any of claims 66-68, wherein the weight ratio of the one or more disintegrants to the cenicriviroc or salt thereof is from about 1:4 to about 3:2 based on the weight of free cenicriviroc.

70. The composition of any of claims 66-69, wherein the weight ratio of the one or more disintegrants to the cenicriviroc or salt thereof is about 1:3; about 2:5; about 1:2; or about 1:1 based on the weight of free cenicriviroc.

71. The composition of any of claims 66-70, wherein the one or more disintegrants are present in an amount of from about 3% to about 9% by weight of the composition.

72. The composition of any of claims 49-71, further comprising one or more lubricants.

73. The composition of claim 72, wherein the one or more lubricants are selected from stearin, magnesium stearate, and stearic acid.

74. The composition of claim 72 or 73, wherein the one or more lubricants is magnesium stearate.

75. The composition of any of claims 72-74, wherein the one or more lubricants are present in an amount of from about 0.5% to about 4% by weight of the composition.

76. The composition of any of claims 49-75, wherein the composition is substantially similar to that of Table 18, 19, 20, 21, 22, 23, or 24.

77. The composition of any of claims 49-76, wherein the composition has a water content of no more than about 4.0% by weight after about four weeks of exposure to about 40° C. at about 75% relative humidity when packaged with a desiccant in a container.

78. The composition of any of claims 49-77, wherein the composition has a water content of no more than about 2.0% by weight after about four weeks of exposure to about 40° C. at about 75% relative humidity when packaged with a desiccant in a container.

79. The composition of any of claims 49-78, wherein the composition has a total impurity and degradant level of no more than about 4.0% after 9 weeks of exposure to about 40° C. at about 75% when packaged with a desiccant in a container.

80. The composition of any of claims 49-79, wherein the composition has a total impurity and degradant level of no more than about 2.0% after 9 weeks of exposure to about 40° C. at about 75% when packaged with a desiccant in a container.

81. The composition of any of claims 49-80, further comprising efavirenz.

82. The composition of claim 81, wherein the weight ratio among cenicriviroc or salt thereof, lamivudine, and efavirenz is from about 1:2:4 based on the weight of free cenicriviroc.

83. The composition of claim 81 or 82, comprising
about 10.3% cenicriviroc or salt thereof, about 18.2% lamivudine, and about 36.4% efavirenz; or
about 9.5% cenicriviroc or salt thereof, about 19.1% lamivudine, and about 38.1% efavirenz by weight of the composition and based on the weight of free cenicriviroc.

84. The composition of any of claims 81-83, wherein the composition is substantially similar to that of Table 28 or 29.

85. The composition of any of claims 81-84, wherein the composition has a water content of no more than about 4.0% by weight after about four weeks of exposure to about 40° C. at about 75% relative humidity when packaged with a desiccant in a container.

86. The composition of any of claims 81-84, wherein the composition has a water content of no more than about 2.0% by weight after about four weeks of exposure to about 40° C. at about 75% relative humidity when packaged with a desiccant in a container.

87. The composition of any of claims 81-84, wherein the composition has a total impurity and degradant level of no more than about 4.0% after 9 weeks of exposure to about 40° C. at about 75% when packaged with a desiccant in a container.

88. The composition of any of claims 81-84, wherein the composition has a total impurity and degradant level of no more than about 2.0% after 9 weeks of exposure to about 40° C. at about 75% when packaged with a desiccant in a container.

89. A pharmaceutical formulation comprising the composition of any of claims 1-88.

90. The formulation of claim 89, wherein the composition in the formulation is in the form of a granulate.

91. The formulation of claim 89 or 90, wherein the composition in the formulation is in form of a capsule.

92. The formulation of claim 89 or 90, wherein the composition in the formulation is in form of a sachet.

93. The formulation of claim 89 or 90, wherein the composition in the formulation is a tablet or a component of a tablet.

94. The formulation any of claims 89-93, further comprising one or more pharmaceutically inactive ingredients.

95. The formulation of any of claims 89-94, wherein the composition is in one or more layers of a multi-layer tablet.

96. The formulation of any of claims 89-94, wherein the composition is in a single layer tablet.

97. The formulation of claim 95, wherein the composition is in a bilayer tablet comprising a single core and a layer outside the single core.

98. The formulation of claim 97, wherein the cenicriviroc or salt thereof and fumaric acid are present in the core; and lamivudine is present in the layer outside the single core.

99. The formulation of claim 97, wherein the cenicriviroc or salt thereof, fumaric acid, and lamivudine are present in the core; and efavirenz is present in the layer outside the single core.

100. A formulation of any of claims 89-99, wherein the formulation is substantially similar to that of Table 3a, 36, 18, 19, 20, 21, 22, 23, 24, 28, or 29.

101. A tablet having a composition substantially similar to that of Table 3a, 36, 18, 19, 20, 21, 22, 23, 24, 28, or 29.

102. A composition of any of claims 1-88, a formulation of any of claims 89-100, or a tablet of claim 101, which is a coated substrate.

103. A method of preparing a composition of any of claims 1-88, a formulation of any of claims 89-100 or a tablet of claim 101, the method comprising:
admixing cenicriviroc or a salt thereof and fumaric acid to form an admixture; and
dry granulating the admixture.

104. The method of claim 103, wherein the cenicriviroc or salt thereof is cenicriviroc mesylate.

105. The method of claim 103 or 104, further comprising admixing one or more fillers with the cenicriviroc or salt thereof and fumaric acid to form the admixture.

106. The method of any of claim 103-105, wherein the one or more fillers are selected from microcrystalline cellulose, calcium phosphate dibasic, cellulose, lactose, sucrose, mannitol, sorbitol, starch, and calcium carbonate.

107. The method of any of claims 103-106, wherein the one or more fillers is microcrystalline cellulose.

108. The method of any of claims 103-107, further comprising admixing one or more disintegrants with the cenicriviroc or salt thereof and fumaric acid to form the admixture.

109. The method of claim 108, wherein the one or more disintegrants are selected from cross-linked polyvinylpyrrolidone, cross-linked sodium carboxymethyl cellulose, and sodium starch glycolate.

110. The method of claim 108 or 109, wherein the one or more disintegrants is cross linked sodium carboxymethyl cellulose.

111. The method of any of claims 103-110, further comprising admixing one or more lubricants with the cenicriviroc or salt thereof and fumaric acid to form the admixture.

112. The method of claim 111, wherein the one or more lubricants are selected from stearin, magnesium stearate, and stearic acid.

113. The method of claim 111 or 112, wherein the one or more lubricants is magnesium stearate.

114. The method of any of claims 103-113, further comprising compressing the dry granulated admixture into a tablet.

115. The method of any of claims 103-113, further comprising filling a capsule with the dry granulated admixture.

116. The method of any of claims 103-114, further comprising mixing the dry granulated admixture with one or more extragranular materials.

117. The method of claim 116, wherein the one or more extragranular materials are one or more additional pharmaceutically active agents.

118. The method of claim 117, wherein the one or more additional pharmaceutically active agents are one or more additional antiretroviral drugs.

119. The method of claim 118, wherein the one or more additional antiretroviral drugs are selected from CCR5 receptor antagonists, entry inhibitors, nucleoside reverse transcriptase inhibitors, nucleotide reverse transcriptase inhibitors, non-nucleoside reverse transcriptase inhibitors, protease inhibitors, integrase inhibitors, and maturation inhibitors.

120. The method of claim 118 or 119, wherein the one or more additional antiretroviral drugs are selected from maraviroc, lamivudine, efavirenz, raltegravir, vivecon, bevirimat, alpha interferon, zidovudine, abacavir, lopinavir, ritonavir, tenofovir, tenofovir disoproxil, tenofovir prodrugs, emtricitabine, elvitegravir, cobicistat darunavir, atazanavir, rilpivirine, and dolutegravir.

121. The method of claim 118, wherein the additional pharmaceutically active agent is lamivudine.

122. The method of claim 118, wherein the one or more additional pharmaceutically active agents are lamivudine and efavirenz.

123. The method of claim 117, wherein the one or more additional pharmaceutically active agents are one or more immune system suppressing agents

124. The method of any of claim 117 or 123, wherein the one or more additional pharmaceutically active agents are selected from the group consisting of cyclosporine, tacrolimus, prednisolone, hydrocortisone, sirolimus, everolimus, azathioprine, mycophenolic acid, methotrexate, basiliximab, daclizumab, rituximab, anti-thymocyte globulin, and anti-lymphocite globulin.

125. The method of any of claim 117 or 123-124, wherein the one or more additional pharmaceutically active agents are selected from the group consisting of tacrolimus and methotrexate.

126. A method of administering cenicriviroc or a salt thereof to a subject, comprising administering a composition of any of claims 1-88, a formulation of any of claims 89-100, a tablet of claim 101, or a composition produced by the method of any of claims 111-125 to a subject.

127. A method of treating a disease, condition, or disorder in a subject, comprising administering a therapeutically effective amount of a composition of any of claims 1-88, a formulation of any of claims 89-100, a tablet of claim 101, or a composition produced by the method of any of claims 111-125 to a subject.

128. The method of claim 127, wherein the disease, condition, or disorder is a viral infection.

129. The method of claim 127 or 128, wherein the disease, condition, or disorder is a retroviral infection.

130. The method of any of claims 127-129, wherein the disease, condition, or disorder is hepatitis, human immunodeficiency virus, or a sarcoma virus.

131. The method of any of claims 127-130, wherein the disease, condition, or disorder is human immunodeficiency virus.

132. The method of claim 127, wherein the disease, disorder, or condition is inflammation.

133. The method of any of claim 127 or 132 wherein the disease, disorder or condition is graft versus host disease, diabetic inflammation, or cardiovascular inflammation.

134. The method of any of claim 127 or 132-133, wherein the disease, disorder or condition is graft versus host disease or prophylaxis thereof.
